(19) United States
(12) Patent Application Publication
(10) Pub. No.: US 2009/0066224 A1
(43) Pub. Date: Mar. 12, 2009

(54) DICARBAZOLE AROMATIC AMINE POLYMERS AND ELECTRONIC DEVICES

(76) Inventors: Wanglin Yu, Midland, MI (US); Weishi Wu, Midland, MI (US); Michelle L. Hudack, Midland, MI (US); James J. O'Brien, Midland, MI (US)

Correspondence Address: SUGHRUE MION, PLLC, 2100 PENNSYLVANIA AVENUE, N.W., SUITE 800, WASHINGTON, DC, 20037, US

(21) Appl. No.: 11/817,444

(22) PCT Filed: Mar. 2, 2006
(86) PCT No.: PCT/US2006/007196

§ 371 Date: Apr. 3, 2008

Related U.S. Application Data
(60) Provisional application No. 60/661,419, filed on Mar. 4, 2005.

(51) Int. Cl. H01J 1/63; C08F 283/06; C07D 209/82; H01L 51/05; C07D 265/38; H01L 31/04

(52) U.S. Cl. 313/504; 525/417; 548/440; 544/102; 136/263; 257/40; 257/E51.005

(57) ABSTRACT

A conjugated or partially conjugated polymer comprising a structural unit of Formula I: I Wherein Ar1 is an aromatic group which contains one or more heteroatoms, or an aromatic group which comprises one or more fused aromatic or non-aromatic rings, said aromatic group may be substituted or unsubstituted; and R1 is alkyl, alkoxy, and aryl group, cyano, or F.

This application claims the benefit of the provisional application, U.S. Application No. 60/661,419, filed Mar. 4, 2005, which is incorporated herein by reference.

This invention relates to polymeric compositions comprising N,N′-dicarbazole-based aromatic amine moieties and electronic devices comprising such compositions.

### BACKGROUND

Fluorene based conjugated polymers are known to have optoelectronic properties. Several reports have demonstrated blue light emission from fluorene homopolymers e.g., A. W. Grice; D. D. C. Bradley, M. T. Bernius; M. Inbasekaran, W. Wu, E. P. Woo; Appl. Phys. Lett. 1998, 73, and Y. Young and Q. Pei; J. Appl. Phys. 81, 3294 (1997). By incorporating different aromatic functional groups into the polymer chain, fluorene-based conjugated polymers have demonstrated different emissive colors with the emissive spectra spanning the entire visible range (400-700 nm) (M. T. Bernius, M. Inbasekaran, J. O'Brien, W. Wu, Adv. Mater. 2000, 12, 1737). Efficient and stable electroluminescence needs efficient injection of holes and electrons into a light-emitting polymer layer from anode and cathode, respectively. Due to the energy mismatch between the highest occupied molecular orbital (HOMO) of fluorene homopolymers and the work function of the anode, the hole-injection of fluorene homopolymers tends to be inefficient. U.S. Pat. No. 6,309,763, U.S. Pat. No. 6,353,083 and U.S. Pat. No. 5,879,821 teach the incorporation of triarylamines into fluorene-based polymers as holetransporting moieties to improve the electroluminescent properties of fluorene-based polymers. USPAP 2004127666 further discovered that the inclusion of tricyclic arylamine in the main chain of a fluorene-based optoelectronic polymer provides improved conductivity at low voltages as well as excellent device efficiency compared to polyfluorenes having other charge transporting groups such as acyclic triarylamines (see, U.S. Pat. No. 6,353,083).

A need remains to develop optoelectronic materials and devices that exhibit improved efficiency and lifetime, and emit light in a variety of colors. Of special interest is to discover new aromatic amine moieties that are suitable to be incorporated into fluorene-based polymers to offer, for example, deeper blue emission with good hole-injection and hole-transporting properties. N,N′-dicarbazole-based aromatic amines are a well-known class of electronic materials used as hole-injection, hole-transporting, and host materials in small molecule optoelectronic devices. However, N,N′-dicarbazole-based aromatic amines have never been incorporated into the backbone of a conjugated polymer.

### SUMMARY OF THE INVENTION

The present invention is directed to a conjugated polymer comprising an N,N′-dicarbazole-based aromatic amine moiety in the main chain of the polymer that exhibits high hole conductivity and deep blue emission.

More specifically, the instant invention is a conjugated or partially conjugated polymer comprising a structural unit of Formula I in the backbone:

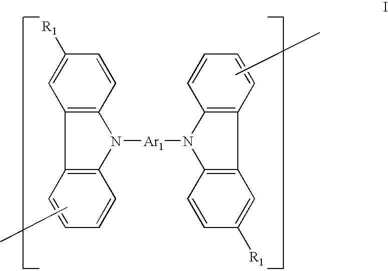

wherein Ar₁ is an aromatic group which contains one or more heteroatoms, or an aromatic group which comprises one or more fused aromatic or non-aromatic rings, said aromatic group may be substituted or unsubstituted; and R₁ is alkyl, alkoxy, an aryl group, cyano, or F.

In another embodiment, the invention is a film of a polymer comprising Formula I. In another aspect, the invention is a blend of a polymer comprising Formula I with at least one additional conjugated polymer. In another embodiment, the invention is an electroluminescent device comprising a film comprising a polymer comprising Formula I. In another embodiment, the invention is a photocell comprising a first electrode, a film comprising the polymer comprising Formula I and a second electrode. In another embodiment, the invention is a field effect transistor comprising: (a) an insulator layer, the insulator layer being an electrical insulator, the insulator layer having a first side and a second side; (b) a gate, the gate being an electrical conductor, the gate being positioned adjacent the first side of the insulator layer; (c) a semiconductor layer, the semiconductor layer comprising the polymer comprising Formula I and a second electrode; (d) a source, the source being an electrical conductor, the source being in electrical contact with the first end of the semiconductor layer; and (e) a drain, the drain being an electrical conductor, the drain being in electrical contact with the second end of the semiconductor layer.

In another aspect, the invention is a compound of Formula IV

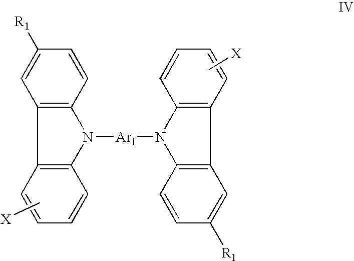

wherein Ar₁ is a substituted or unsubstituted aromatic group which contains one or more heteroatomes or comprises one or more fused aromatic or non-aromatic rings, which rings may be substituted or unsubstituted, R₁ is alkyl, alkoxy, aryl-substituted group, cyano, or F, and X is a halogen or a boronate group.

### DETAILED DESCRIPTION OF THE INVENTION

The instant invention is a conjugated or partially conjugated polymer comprising a structural unit of Formula I:

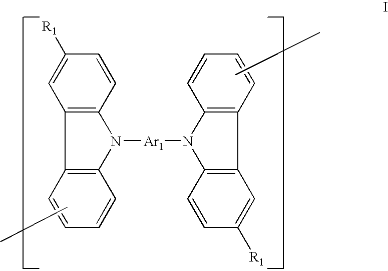

wherein Ar₁ is an aromatic group which contains one or more heteroatoms, or an aromatic group which comprises one or more fused aromatic or non-aromatic rings, said aromatic group may be substituted or unsubstituted; and R₁ is alkyl, alkoxy, an aryl group, cyano, or F.

Preferably, Ar₁ is selected from the group consisting of fluorenyl, thiophenyl, furanyl, pyrrolyl, pyridinyl, naphthalenyl, anthracenyl, phenanthrenyl, tetracenyl, perylenyl, quinolinyl, isoquinolinyl, quinazolinyl, phenanthridenyl, phenanthrolinyl, phenazinyl, acridinyl, dibenzosilolyl, phthalazinyl, cinnolinyl, quinoxalinyl, benzoxazolyl, benzimidazolyl, benzothiophenyl, benzothiazolyl, carbazolyl, benzoxadiazolyl, benzothiadiazolyl, thieno[3,4-b]pyrazinyl, [1,2,5]thiadiazolo[3,4-g]-quinoxalinyl, benzo[1,2-c; 3-4-c′]bis[1,2,5]-thiadiazolyl, pyrazino[2,3-g]quinoxalinyl, benzofuranyl, indolyl, dibenzofuranyl, dibenzothiophenyl, thianthrenyl, benzodioxinyl, benzodioxanyl, dibenzodioxinyl, phenazinyl, phenoxathiinyl, benzodithiinyl, benzodioxolyl, benzocyclobutenyl, dihydrobenzodithiinyl, dihydrothienodioxinyl, chromanyl, isochromanyl, 9,10-dihydrophenanthrenyl, thiazinyl, phenoxazinyl, isoindolyl, dibenzothiophenesulfonyl, and phenothiazinyl, or Ar₁ is selected from the group consisting of phenyl, biphenyl, a 9,9-disubstituted-2,7-fluorenyl, N-substituted-3,6-carbazolyl, N-substituted-3,7-phenoxazinyl, N-substituted-3,7-phenothiazinyl.

In another embodiment, the instant invention is a conjugated or partially conjugated polymer comprising a structural unit of Formula I;

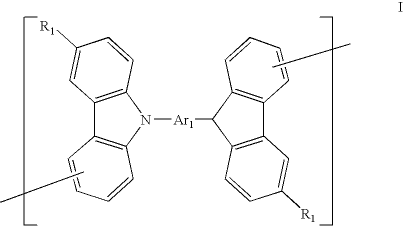

wherein Ar₁ is biphenyl which may be substituted or unsubstituted, and R₁ is alkyl, alkoxy, an aryl group, cyano, or F.

In both embodiments, R₁ can independently be alkyl, alkoxyl, an aryl group, cyano, or F. Preferably, R₁ is a C₁-C₂₀ alkyl group, a carbo-C₁-C₂₀-alkoxy group, a C₁-C₂₀-alkoxy group, which may contain one or more heteroatoms, such as O, S, N, or Si, and in which one or more hydrogen atoms may be replaced by F, or aromatic groups, or a C₆-C₄₀ aryl group which may be further substituted and which may contain one or more heteroatoms. More preferably, R₁ is methyl, ethyl, propyl, butyl, methoxy, ethoxy, propoxy, butoxy, phenyl, or tolyl.

In another embodiment, the instant invention is a conjugated or partially conjugated polymer comprising a structural unit of Formula II:

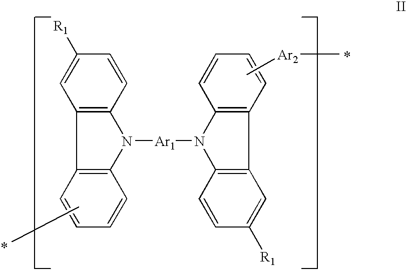

wherein Ar₁ is an aromatic group and Ar₂ is another aromatic group which may be the same or different with Ar₁, both Ar₁ and Ar₂ may be substituted or unsubstituted; Ar₁ contains one or more heteroatomes or comprises one or more fused aromatic or non-aromatic rings, which rings may be substituted or unsubstituted, and R₁ is alkyl, alkoxy, aryl-substituted group, cyano, or F.

In another embodiment, the instant invention is a conjugated or partially conjugated polymer comprising a structural unit of Formula II;

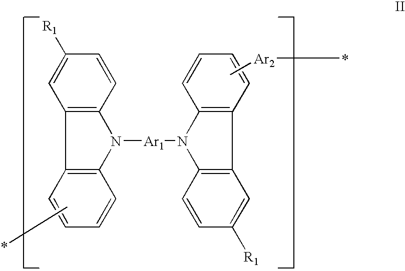

wherein Ar₁ is biphenyl and Ar₂ is another aromatic group which may be the same or different with Ar₁, both Ar₁ and Ar₂ may be substituted or unsubstituted, and R₁ is alkyl, alkoxy, an aryl group, cyano, or F.

In both embodiments, preferably, Ar₂ is a 1,4-phenylene, a 1,3-phenylene, a 1,2-phenylene, a 4,4′-biphenylene, a naphthalene-1,4-diyl, a naphthalene-2,6-diyl, a furan-2,5-diyl, a thiophene-2,5-diyl, a 2,2′-bithiophene-5,5-diyl, an anthracene-9,10-diyl, a 2,1,3-benzothiadiazole-4,7-diyl, an N-substituted carbazole-3,6-diyl, an N-substituted carbazole-2,7-diyl, a dibenzosilole-3,8-diyl, a dibenzosilole-4,7-diyl, an N-substituted-phenothiazine-3,7-diyl, an N-substituted-phenoxazine-3,7-diyl, a triarylamine-diyl including a triphenylamine-4,4′-diyl, a diphenyl-p-tolylamine-4,4′-diyl, and an N,N-diphenylaniline-3,5-diyl, an N,N,N′,N′-tetraaryl-1,4-diaminobenzene-diyl, an N,N,N′,N′-tetraarylbenzidine-diyl, an arylsilane-diyl, and an 9,9-disubstituted fluorene-2,7-diyl.

More preferably, Ar₂ comprises a fluorene having the Formula III

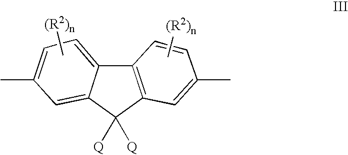

where Q is R′ or Ar, wherein Ar is an aryl or heteroaryl group of C₄ to C₄₀ or substituted aryl or heteroaryl group of C₄ to C₄₀; R′ is independently, in each occurrence, H, C₁₋₄₀ hydrocarbyl or C₃₋₄₀ hydrocarbyl containing one or more S, N, O, P or Si atoms, or both of R′ together with the 9-carbon on the fluorene may form a C₅₋₂₀ ring structure that may contain one or more S, N, Si, P or O atoms; R² is independently in each occurrence a C₁₋₄₀ hydrocarbon, C₃₋₄₀ hydrocarbyl containing one or more heteroatoms of S, N, O, P or Si, or a substituted or unsubstituted aryl group or heteroaryl group; n is independently in each occurrence, 0-3.

The polymer of the instant invention represented by the formula (I) preferably comprises independently in each occurrence a moiety in the polymer chain selected from the group of conjugated units of the formulas or a combination of the formulas:

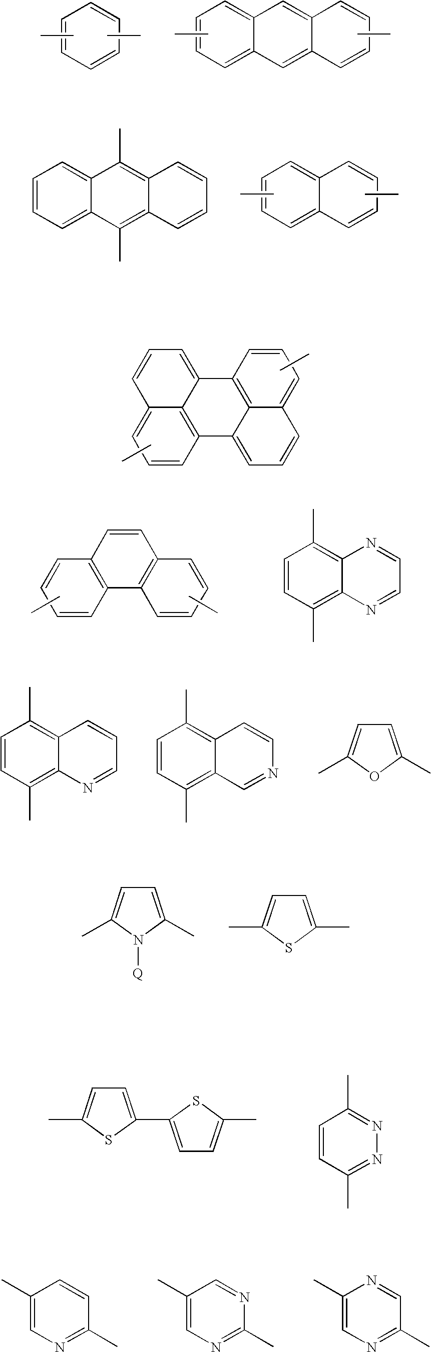

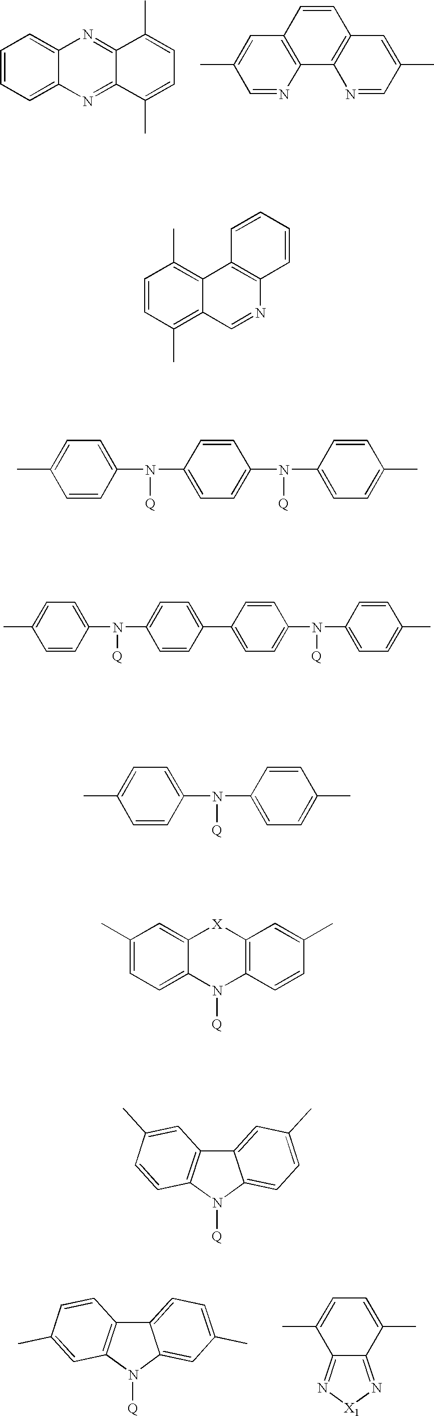

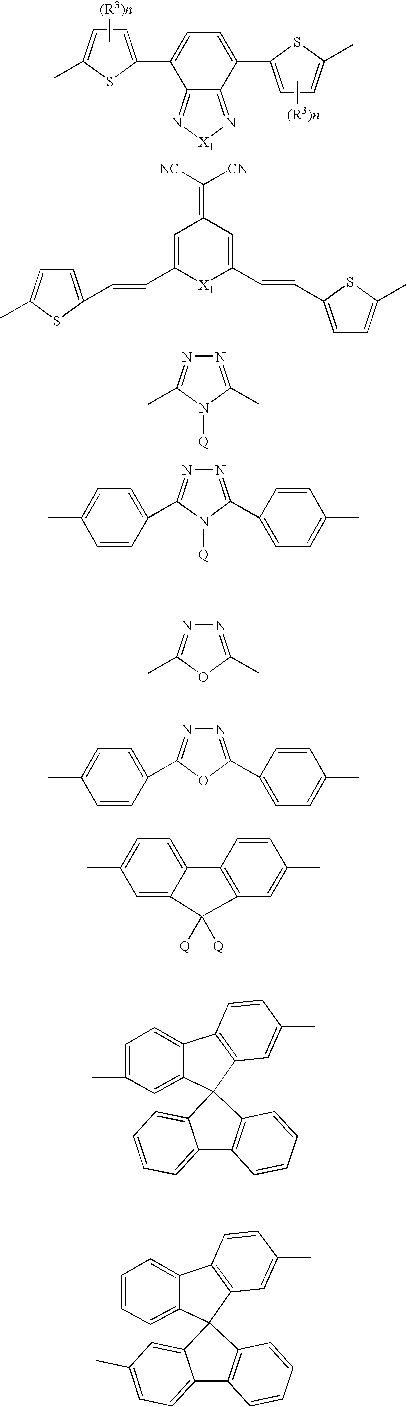

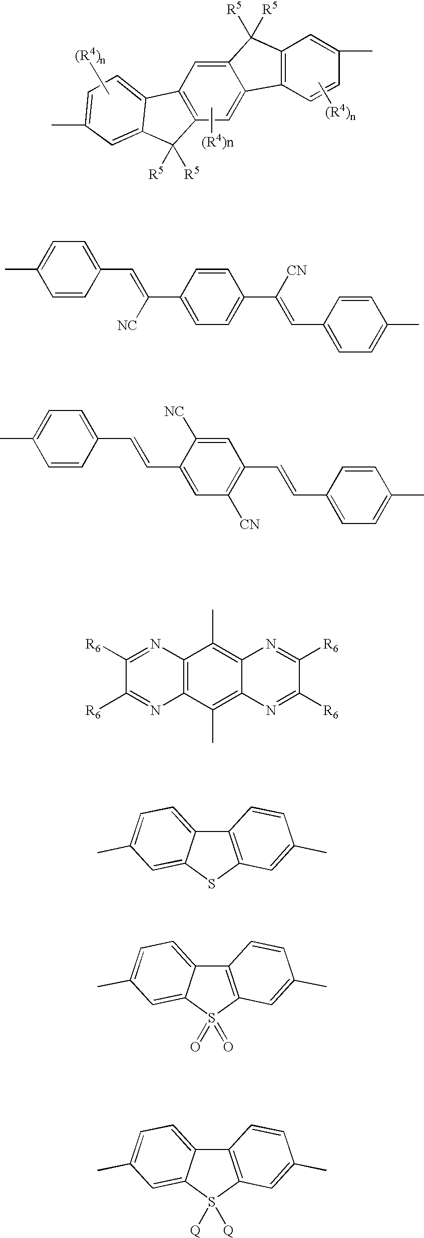

wherein the conjugated units may bear one or more substitutents, such substituents being independently, in each occurrence, C₁₋₂₀ hydrocarbyl, C₁₋₂₀ hydrocarboxyloxy, C₁₋₂₀ thioether, C₁₋₂₀ hydrocarbyloxycarbonyl, C₁₋₂₀ hydrocarboxycarbonyloxy, cyano, or fluoro group;

### X₁ is O or S;

### Q is R′ or Ar;

R³ is independently, in each occurrence, C₁₋₂₀ hydrocarbyl, C₁₋₂₀ hydrocarbyloxy, C₁₋₂₀ thioether, C₁₋₂₀ hydrocarbyloxycarbonyl, C₁₋₂₀ hydrocarbylcarbonyloxy, cyano or fluoro group; R⁴ is independently, in each occurrence, H, C₁₋₄₀ hydrocarbyl or C₃₋₄₀ hydrocarbyl containing one or more S, N, O, P or Si atoms, or both of R⁴ together with the 9-carbon on the fluorene may form a C₅₋₂₀ ring structure that may contain one or more S, N, Si, P or O atoms; and R⁵ is independently, in each occurrence, H, C₁₋₄₀ hydrocarbyl or C₃₋₄₀ hydrocarbyl containing one or more S, N, O, P or Si atoms;
n is independently in each occurrence, 0-3;
Ar is an aryl or heteroaryl group of C₄ to C₄₀ or substituted aryl or heteroaryl group of C₄ to C₄₀; R′ is independently, in each occurrence, H, C₁₋₄₀ hydrocarbyl or C₃₋₄₀ hydrocarbyl containing one or more S, N, O, P or Si atoms, or both of R′ together with the 9-carbon on the fluorene may form a C₅₋₂₀ ring structure that may contain one or more S, N, Si, P or O atoms.

The instant invention includes an electroluminescent device comprising at least one organic film comprising the polymer comprising Formula I, arranged between an anode material and a cathode material such that under an applied voltage, the organic film emits light which is transmitted through a transparent exterior portion of the device.

The instant invention is also a field effect transistor comprising: (a) an insulator layer, the insulator layer being an electrical insulator, the insulator layer having a first side and a second side; (b) a gate, the gate being an electrical conductor, the gate being positioned adjacent the first side of the insulator layer; (c) a semiconductor layer, the semiconductor layer comprising the polymer comprising Formula I and a second electrode; (d) a source, the source being an electrical conductor, the source being in electrical contact with the first end of the semiconductor layer; and (e) a drain, the drain being an electrical conductor, the drain being in electrical contact with the second end of the semiconductor layer. The instant invention also includes a photocell comprising a first electrode, a film comprising the polymer comprising Formula I and a second electrode.

In another embodiment, the instant invention is a compound of Formula IV

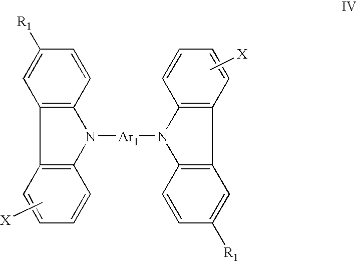

wherein Ar₁ is a substituted or unsubstituted aromatic group which contains one or more heteroatomes or comprises one or more fused aromatic or non-aromatic rings, which rings may be substituted or unsubstituted, R₁ is alkyl, alkoxy, aryl-substituted group, cyano, or F, and X is a halogen or a boronate group.

Preferably, Ar₁ is selected from the group consisting of fluorenyl, thiophenyl, furanyl, pyrrolyl, pyridinyl, naphthalenyl, anthracenyl, phenanthrenyl, tetracenyl, perylenyl, quinolinyl, isoquinolinyl, quinazolinyl, phenanthridenyl, phenanthrolinyl, phenazinyl, acridinyl, dibenzosilolyl, phthalazinyl, cinnolinyl, quinoxalinyl, benzoxazolyl, benzimidazolyl, benzothiophenyl, benzothiazolyl, carbazolyl, benzoxadiazolyl, benzothiadiazolyl, thieno[3,4-b]pyrazinyl, [1,2,5]thiadiazolo[3,4-g]-quinoxalinyl, benzo[1,2-c; 3-4-c′]bis[1,2,5]-thiadiazolyl, pyrazino[2,3-g]quinoxalinyl, benzofuranyl, indolyl, dibenzofuranyl, dibenzothiophenyl, thianthrenyl, benzodioxinyl, benzodioxanyl, dibenzodioxinyl, phenazinyl, phenoxathiinyl, benzodithiinyl, benzodioxolyl, benzocyclobutenyl, dihydrobenzodithiinyl, dihydrothienodioxinyl, chromanyl, isochromanyl, 9,10-dihydrophenanthrenyl, thiazinyl, phenoxazinyl, isoindolyl, dibenzothiophenesulfonyl, and phenothiazinyl, or Ar₁ is selected from the group consisting of phenyl, biphenyl, a 9,9-disubstituted-2,7-fluorenyl, N-substituted-3,6-carbazolyl, N-substituted-3,7-phenoxazinyl, N-substituted-3,7-phenothiazinyl.

In another embodiment, the instant invention is a compound of Formula IV

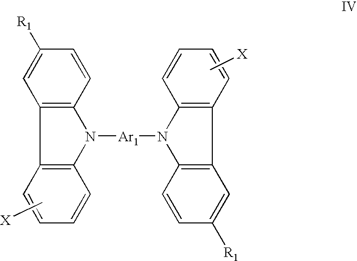

wherein Ar₁ is substituted or unsubstituted biphenyl, R₁ is alkyl, alkoxy, an aryl group, cyano, or F, and X is a halogen or a boronate group.

In both embodiments, preferably, X is bromine.

R₁ is independently alkyl, alkoxyl, aryl-substituted group, cyano, or F. R₁ preferable R1 is a C₁-C₂₀ alkyl group, a carbo-C₁-C₂₀-alkoxy group, a C₁-C₂₀-alkoxy group, which may contain one or more heteroatoms, such as O, S, N, P, or Si, and in which one or more hydrogen atoms may be replaced by F, or aromatic groups, or a C₆-C₄₀ aryl group which may be further substituted and which may contain one or more heteroatoms. More preferably, R₁ is methyl, ethyl, propyl, butyl, methoxy, ethoxy, propoxy, butoxy, phenyl or tolyl.

The polymers of the invention have a weight average molecular weight of about 10,000 Daltons or greater, 20,000 Daltons or greater, or preferably 50,000 Daltons or greater; 1,000,000 Daltons or less, 500,000 Daltons or less, and preferably 400,000 Daltons or less. Molecular weights are determined using gel permeation chromatography using polystyrene as an internal standard.

The polymers demonstrate a polydispersity (Mw/Mn) of 10 or less, 5 or less, 4 or less and preferably 3 or less.

The polymers of this invention may be assembled by any known coupling reaction for making aromatic compounds. Preferably, the Suzuki coupling reaction is used. The Suzuki reaction couples aromatic compounds using a diboronated aromatic moiety and a dihalogenated aromatic moiety. The reaction allows for the creation of long chain, high molecular weight polymers. Additionally, by controlling the sequence of addition, either random or block copolymers may be produced.

Preferably, the Suzuki reaction starts with a diboronated monomer. The Suzuki process is taught in U.S. Pat. No. 5,777,070, which is expressly incorporated herein by reference.

Toluene or xylenes are the preferred solvents for the Suzuki reaction to prepare the polymers of the instant invention. Sodium carbonate in water is the preferred base, a palladium complex catalyst, such as tetrakis(triphenylphosphine)palladium or dichlorobis(triphenylphosphine)palladium(II) is the preferred catalyst, and a phase transfer catalyst, preferably, a quaternary ammonium salt, is used to speed up the reaction for achieving high molecular weight in a short period of time.

A general synthetic route is outlined in the following scheme to illustrate the synthetic art of a monomer of Formula II of the instant invention. Starting materials for synthesizing the monomer(s) of Formula II (wherein, Y is a halogen atom, Ar₁, R₁, and X are defined as above) of the instant invention, are commercially available from many commercial vendors such as Aldrich Chemical Company.

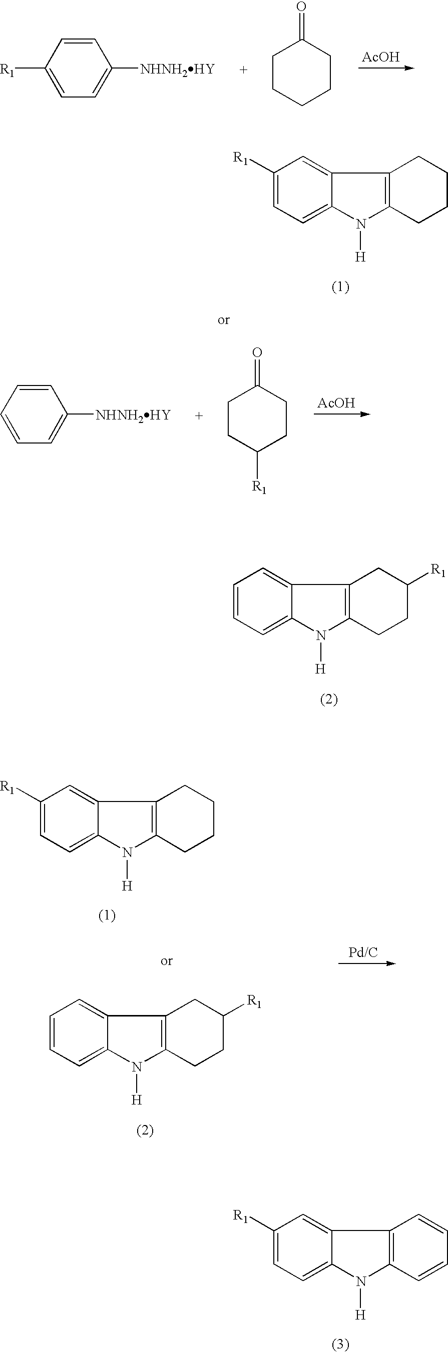

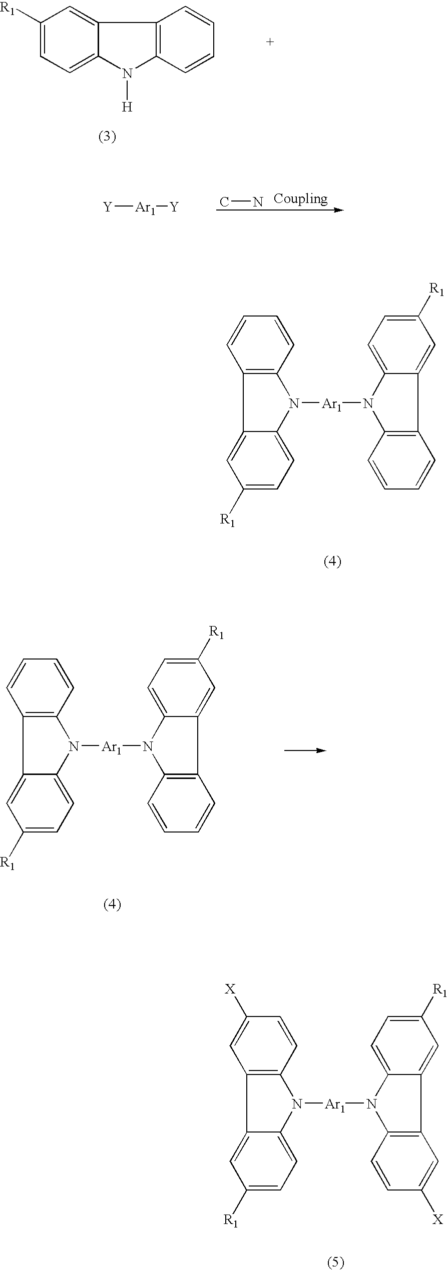

3-Substituted-9-H-carbazole (3) may be prepared following a literature procedure (A. R. Katritzky and Z. Wang, Journal of Heterocyclic Chemistry, 25, 671, (1988); J.-K Luo, R. N. Castle, M. L. Lee, Journal of Heterocyclic Chemistry, 26, 1213, (1989); Crosby U. Rogers and B. B. Corson, Journal of the American Chemical Society, 69, 2910) starting from a para-substituted or unsubstituted phenylhydrazine hydrohalogenide salt and cyclohexanone or a substituted cyclohexanone. For example, p-tolylhydrazine hydrochloride reacts with cyclohexanone in boiling glacial acetic acid to produce 1,2,3,4-tetrahydro-6-methylcarbazole. The dehydrogenation of 1,2,3,4-tetrahydro-6-methylcarbazole catalyzed by palladium on charcoal at elevated temperatures generates 3-methyl-9-H-carbazole as one example of compound (3). 3-methyl-9-H-carbazole can also be prepared starting from phenylhydrazine hydrochloride and 4-methyl-cyclohexanone using the similar reactions, in which the intermediate compound (2) is 1,2,3,4-tetrahydro-3-methylcarbazole. A bis(3-substitute-carbazolyl) aromatic compound (4) can be prepared by reacting a compound (3) with a dihalogenated aromatic compound, for example, 1,4-diiodobenzene, 4,4′-diiodobiphenyl, 3,6-dibromo-9-p-tolylcarbazole, through a C—N coupling reaction. Useful C—N coupling reactions include and are not limited to the Ullmann reaction (U.S. Pat. No. 4,588,666) and palladium-catalyzed cross coupling reactions (M. S. Driver, J. F. Hartwig, Journal of the American Chemical Society, 118, 7217 (1996); J. P. Wolfe, S. Wagaw, S. L. Buchwald, Journal of the American Chemical Society, 118, 7215 (1996); J. P. Wolfe and S. L. Buchwald, Journal of Organic Chemistry, 61, 1133 (1996); A. S. Guran, R. A. Rennels, S. L. Buchwald, Angew. Chem. Int. Ed. Engl., 34, 1348 (1995); J. Louie, J. F. Hartwig, Tetrahedron Lett., 36, 3609 (1995)). A dihalogenated monomer of compound (5) can be prepared by treating a compound (4) with a halogenation reagent, such as N-bromosuccinimide (NBS), N-iodosuccinimide (NIS), bromine. A dihalogenated monomer of compound (5) may be further converted to a diboronate monomer using a known art (U.S. Pat. No. 6,169,163; W.-L. Yu, J. Pei, Y. Cao, A. J. Heeger, Chemical Communications, 1837 (1999); T. Tshiyama, M. Murata, N. Miyaura, Journal of Organic Chemistry, 60, 7508 (1995)).

Another aspect of this invention is related to polymer blends. The blends comprise a polymer containing structural units of Formula I or Formula I blended with at least one other conjugated polymer, preferably a conjugated polymer. As used herein, the term “conjugated polymer” means a polymer with a backbone of overlapping n orbitals. Conjugated polymers that may be used in the blends include polyfluorenes, poly(arylenevinylene), polyphenylenes, polyindenofluorenes and polythiophenes, including homopolymers, co-polymers or substituted homopolymers and/or copolymers of any of these conjugated polymers.

Preferably, the polymer blend is composed of at least 1 weight percent of a polymer containing a structural unit of Formula I. The most preferred polymer blends have high photoluminescent and electroluminescent efficiency. Other additives such as viscosity modifiers, antioxidants and coating improvers may optionally be added. Additionally, blends of two or more low polydispersity polymers of similar compositions but different molecular weight can also be formulated.

Another aspect of this invention is the films formed from the polymers of the invention. Such films can be used in polymeric light emitting diodes, photovoltaic cells and field effect transistors. Preferably, such films are used as emitting layers or charge carrier transport layers. The films may also be used as protective coatings for electronic devices and as fluorescent coatings. The thickness of the film or coating is dependent upon the use.

Generally, such thickness can be from 0.005 to 200 microns. When the coating is used as a fluorescent coating, the coating or film thickness is from 50 to 200 microns. When the coatings are used as electronic protective layers, the thickness of the coating can be from 5 to 20 microns. When the coatings are used in a polymeric light-emitting diode, the thickness of the layer formed is 0.005 to 0.2 microns. The polymers of the invention form good pinhole-free and defect-free films.

The films are readily formed by coating the polymer composition from another embodiment of this invention in which the composition comprises the polymer and at least one organic solvent. Preferred solvents are aliphatic hydrocarbons, chlorinated hydrocarbons, aromatic hydrocarbons, ketones, ethers and mixtures thereof. Additional solvents which can be used include 1,2,4-trimethylbenzene, 1,2,3,4-tetramethyl benzene, pentylbenzene, mesitylene, cumene, cymene, cyclohexylbenzene, diethylbenzene, tetralin, decalin, 2,6-lutidine, 2-fluoro-m-xylene, 3-fluoro-o-xylene, 2-chlorobenzotrifluoride, dimethylformamide, 2-chloro-6-fluorotoluene, 2-fluoroanisole, anisole, 2,3-dimethylpyrazine, 4-fluoroanisole, 3-fluoroanisole, 3-trifluoro-methylanisole, 2-methylanisole, phenetole, 4-methylanisole, 3-methylanisole, 4-fluoro-3-methylanisole, 2-fluorobenzonitrile, 4-fluoroveratrol, 2,6-dimethylanisole, 3-fluorobenzonitrile, 2,5-dimethylanisole, 2,4-dimethylanisole, benzonitrile, 3,5-dimethylanisole, N,N-dimethylaniline, ethyl benzoate, 1-fluoro-3,5-dimethoxybenzene, 1-methylnaphthalene, N-methylpyrrolidinone, 3-fluorobenzotrifluoride, benzotrifluoride, benzotrifluoride, dioxane, trifluoromethoxybenzene, 4-fluorobenzotrifluoride, 3-fluoropyridine, toluene, 2-fluorotoluene, 2-fluorobenzotrifluoride, 3-fluorotoluene, 4-isopropylbiphenyl, phenyl ether, pyridine, 4-fluorotoluene, 2,5-difluorotoluene, 1-chloro-2,4-difluorobenzene, 2-fluoropyridine, 3-chlorofluorobenzene, 3-chlorofluorobenzene, 1-chloro-2,5-difluorobenzene, 4-chlorofluorobenzene, chlorobenzene, o-dichlorobenzene, 2-chlorofluorobenzene, p-xylene, m-xylene, o-xylene or mixture of o-, m-, and p-isomers. It is preferable that such solvents have relatively low polarity. High boilers and solvent mixtures are better for ink jetting, but xylenes and toluene are best for spin coating. Preferably, the solution contains from about 0.1 to 5 percent of a polymer comprising a structural unit of Formula I. Films can be prepared by means well known in the art including spin-coating, spray-coating, dip-coating, roll-coating, offset printing, ink jet printing, screen printing, stamp-coating or doctor blading.

In a preferred embodiment, the invention is a composition comprising a polymer or polymer blend of the invention in a solvent. Solvents which can be used include toluene, xylene, a mixture of o, m and p-isomers of xylene, mesitylene, diethylbenzene, ethylbenzene or benzene derivatives of higher substituted level. Preferably, the solution contains from 0.1 to 10 weight percent of the composition. For thin coatings, it is preferred that the composition contains from 0.5 to 5.0 percent by weight of the composition. The composition is applied to the appropriate substrate by the desired method and the solvent is allowed to evaporate. Residual solvent may be removed by vacuum, heat and/or by sweeping with an inert gas such as nitrogen.

The polymers of this invention demonstrate strong electroluminesence in addition to photoluminesence. Thus, another aspect of the invention relates to organic electroluminescent (EL) devices having a film comprising the polymers of this invention. Preferably, the EL devices of this invention emit light when subjected to an applied voltage of 20 volts or less, 10 volts or less and preferably 6 volts or less.

An organic EL device typically consists of an organic film sandwiched between an anode and a cathode. When a positive bias is applied to the device, holes are injected into the organic film from the anode, and electrons are injected into the organic film from the cathode. The combination of a hole and an electron may give rise to an exciton that may undergo radiative decay to the ground state by liberating a photon.

In practice, the anode is commonly a mixed oxide of tin and indium for its conductivity and transparency. The mixed oxide (ITO) is deposited on a transparent substrate such as glass or plastic so that the light emitted by the organic film may be observed. The organic film may be the composite of several individual layers each designed for a distinct function. Because holes are injected from the anode, the layer next to the anode should have the functionality of transporting holes. Similarly, the layer next to the cathode should have the functionality of transporting electrons. In many instances, the electron or hole transporting layer may also act as the emitting layer. In some instances, a single layer may perform the combined functions of hole and electron transport and light emission.

The metallic cathode may be deposited either by thermal evaporation or by sputtering. The thickness of the cathode may be from 1 nm to 1000 nm. The preferred metals are calcium, magnesium, indium, aluminum and barium. A thin layer (1-10 nm) of an alkali or alkaline metal halide, e.g., LiF, NaF, CsF or RbF, may be used as a buffering layer between the light emitting polymer and the cathode, calcium, barium, or magnesium. Alloys of these metals may also be used. Alloys of aluminum containing 1 to 5 percent of lithium and alloys of magnesium containing at least 80 percent of magnesium are preferred.

In another embodiment, the electroluminescent device comprises at least one hole injecting polymer film (PEDOT film, for example) and a light-emitting polymer film comprised of the composition of the invention, arranged between an anode material and a cathode material such that under an applied voltage, holes are injected from the anode material into the light emitting polymer via the hole-injecting polymer film and electrons are injected from the cathode material into the light-emitting polymer film when the device is forward biased, resulting in light emission from the light-emitting layer. In another embodiment, layers of hole-transporting polymers are arranged so that the layer closest to the anode has the lowest oxidation potential, with the adjacent layers having progressively higher oxidation potentials. By these methods, electroluminescent devices having relatively high light output per unit voltage may be prepared.

Another embodiment of the invention relates to photocells comprising one or more of the polymers of the invention wherein the polymers are present as single-layer films or as multiple-layer films, whose combined thickness is in the range of 10 nm to 1000 nm, in the range of 25 nm to 500 nm, preferably in the range of 50 nm to 300 nm. When two or more polymers are used, they may be deposited separately as distinct layers or deposited as one layer from a solution containing a blend of the desired polymers.

“Photocells” mean a class of optoelectronic devices that can convert incident light energy into electrical energy. Examples of photocells are photovoltaic devices, solar cells, photodiodes, and photodetectors. A photocell generally comprises a transparent or semi-transparent first electrode deposited on a transparent substrate. A polymer film is then formed onto the first electrode that is, in turn, coated by a second electrode. Incident light transmitted through the substrate and the first electrode is converted by the polymer film into excitons that can dissociate into electrons and holes under the appropriate circumstances, thus, generating an electric current.

Another embodiment of the invention relates to metal-insulator-semiconductor field effect transistors comprising one or more of the polymers of the invention which serve as a semiconducting polymer. A field effect transistor comprises five elements. The first element is an insulator layer. The insulator layer is an electrical insulator, having a first side and a second side. The second element is a gate. The gate is an electrical conductor. The gate is positioned adjacent the first side of the insulator layer.

The third element is a semiconductor layer. The semiconductor layer comprises a polymer comprising a structural unit of Formula I above. The semiconductor layer has a first side, a second side, a first end and a second end, the second side of the semiconductor layer being adjacent to the second side of the insulator layer. The polymer is deposited onto an insulator wherein the polymers are present as single-layer films or as multiple-layer films whose combined thickness is in the range of 10 nm to 1000 nm, in the range of 25 nm to 500 nm, preferably in the range of 50 nm to 300 nm.

The fourth element of a field effect transistor is a source. The source is an electrical conductor. The source is in electrical contact with the first end of the semiconductor layer. The fifth element is a drain. The drain is an electrical conductor. The drain is in electrical contact with the second end of the semiconductor layer. A negative voltage bias applied to the gate causes the formation of a hole conduction channel in the semiconductor layer connecting the source to the drain. A positive bias applied to the gate causes the formation of an electron-conducting channel in the semiconductor layer.

As with electroluminiscent devices, the polymer films comprising the semiconductor layer may be formed by solvent-based processing techniques such as spin-coating, roller-coating, dip-coating, spray-coating and doctor-blading and ink jet printing. When two or more polymers are used, they may be deposited separately as distinct layers or deposited as one layer from a solution containing a blend of the desired polymers.

Two electrodes (source and drain) are attached to the semiconducting polymer and a third electrode (gate) onto the opposite surface of the insulator. If the semiconducting polymer is hole transporting (i.e, the majority carriers are positive holes), then applying a negative DC voltage to the gate electrode induces an accumulation of holes near the polymer-insulator interface, creating a conduction channel through which electric current can flow between the source and the drain. The transistor is in the “on” state. Reversing the gate voltage causes a depletion of holes in the accumulation zone and cessation of current. The transistor is in the “off” state.

### EXAMPLES

The following examples are included for illustrative purpose and do not limit the scope of the claims.

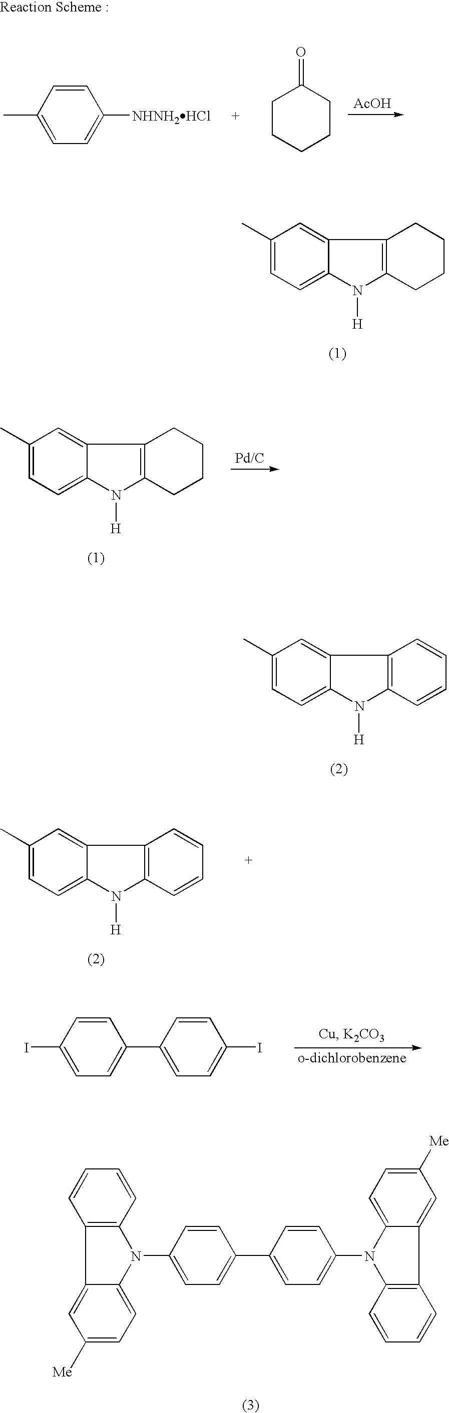

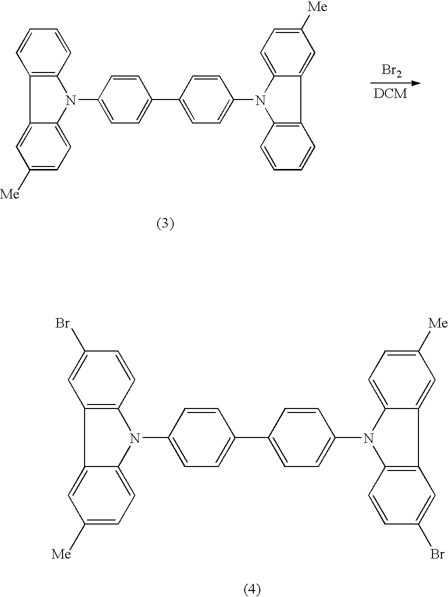

### Synthesis of 1,2,3,4-Tetrahydro-6-methylcarbazole (Compound 1)

Cyclohexanone (49.1 g, 0.5 moles) and glacial acetic acid (180 g) were charged into a 1 L 3-neck round bottom flask (RBF). The solution was heated to reflux. p-Tolylhydrazine hydrochloride (79.3 g, 0.3 moles) was added under reflux during a period of 1 hour. After the addition of p-tolylhydrazine hydrochloride, the reflux was continued for another 3 hours under nitrogen. The heating mantle was removed and the reaction mixture was cooled and then filtered. The solids were washed with water and then with 300 mL of 75% methanol and filtered. The crude product, collected via filtration, was off-white crystals. The crude product was purified by re-crystallization from methanol to give 70 g of colorless needle crystals as the final product. HPLC indicated a purity of essentially 100 wt %.

### Synthesis of 3-Methylcarbazole (Compound 2)

1,2,3,4-Tetrahydro-6-methylcarbazole (Compound 1) (35 g) and 5% palladium charcoal (12 g) in a 1 L RBF were heated at 260° C. under nitrogen for 1.5 hour. After cooling down to room temperature, THF was added to dissolve the compounds. The charcoal and Pd were removed by filtration. THF was removed and then the crude product was re-crystallized from ethanol twice. 25.9 g of the final product were obtained as white crystals. HPLC indicated a purity of 99.5 wt %.

### Synthesis 4,4′-Bis(3-methylcarbazol-9-yl)biphenyl (Compound 3)

10 g (55.2 mmol) of 3-methylcarbazole (Compound 2), 7.47 g (18.4 mmol) of 4,4′-diiodobiphenyl, 7.1 g (110.4 mmol) of copper, 30.4 g (0.22 mol) of potassium carbonate, and 1.45 g (5.5 mmol) of 18-crown-6 were dispersed in 270 mL of o-dichlorobenzene under nitrogen in a 500 mL 3-neck RBF equipped with condenser and Dean Stark trap. The suspension was degassed with nitrogen for 15 minutes and then heated to reflux under nitrogen. The water generated during the reaction was removed through the Dean Stark trap. After 16 hours, HPLC showed no starting material of 4,4′-diiodobiphenyl. The reflux was continued for another 4 hours before removing the heating mantle. After the reaction was cooled to near room temperature, the reaction mixture was filtered though a basic alumina bed (˜2 cm thick) and eluted with toluene. The solvents were removed on a rotary evaporator under reduced pressure to give white solid as the crude product. The product was purified by re-crystallization from toluene to give white crystals. HPLC indicated a purity of 99.3 wt %. The product was dried in vacuum oven at 55° C. for 2 hours. 8.75 g of white crystals were obtained as the final product.

### Synthesis of 4,4′-Bis(3-bromo-6-methylcarbazol-9-yl)biphenyl (Compound 4)

To a solution of 3 g (5.85 mmol) of 4,4′-bis(3-methylcarbazol-9-yl)biphenyl (Compound 3) in 500 mL of dichloromethane was added dropwise 1.97 g (12.28 mmol) of bromine in 50 mL of dichloromethane at room temperature. After the addition, the reaction was stirred at room temperature for another 1 h. The mixture was filtered and the crude product was collected by filtration as white solids. Two re-crystallizations from toluene produced the final product as white crystals (0.78 g). HPLC showed a purity of 99.1 wt %.

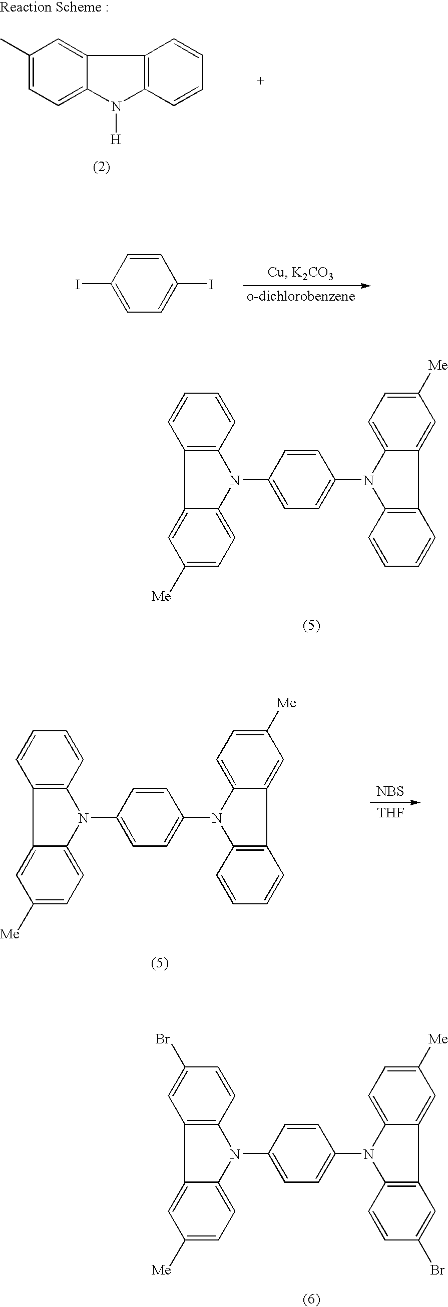

### Synthesis of 1,4-Bis(3-methylcarbazol-9-yl)benzene (Compound 5)

7.25 g (40 mmol) of 3-methylcarbazole (Compound 2), 4.95 g (15 mmol) of 1,4-diiodobenzene, 6.4 g (0.1 mmol) of copper, 28 g (0.22 mol) of potassium carbonate, and 1.3 g (5.5 mmol) of 18-crown-6 were dispersed in 250 mL of o-dichlorobenzene under nitrogen in a 500 mL 3-neck RBF equipped with condenser and Dean Stark trap. After an overnight reaction, HPLC showed no starting material of 1,4-diiodobenzene. The reflux was continued for another 4 hour before removing the heating mantle. After the reaction was cooled to room temperature, the reaction mixture was filtered through a basic alumina bed (˜2 cm thick) and eluted with toluene. The solvents were removed on a rotary evaporator under reduced pressure to give white solid as the crude product. The product was purified by re-crystallization from toluene to give white crystals. HPLC indicated a purity of 99.6 wt %. The product was dried in a vacuum oven at 55° C. overnight. 6.01 g of white crystals were obtained as the final product. Purity was 99.6 wt % by HPLC. The yield was 91.6 mole %.

### Synthesis of 1,4-Bis(3-bromo-6-methylcarbazol-9-yl)benzene (Compound 6)

To a solution of 4.5 g (10.3 mmol) of 1,4-bis(3-methylcarbazol-9-yl)benzene (Compound 5) dissolved in ˜700 mL of THF was added 3.71 g (20.8 mmol) of NBS dissolved in ˜10 mL of DMF at room temperature. The reaction mixture was stirred at room temperature overnight. White solids precipitated. HPLC showed a complete conversion of the starting material. The reaction mixture was heated to reflux and became clear. 1% of NBS was added at room temperature and then heated to reflux. The reaction mixture was heated to reflux and then cooled down to room temperature to recrystallize the product. White needle crystals were obtained. HPLC showed a purity of 99 wt %. Re-crystallization from THF was repeated once and the purity was increased to 99.4 wt %. The yield was 3.0 g.

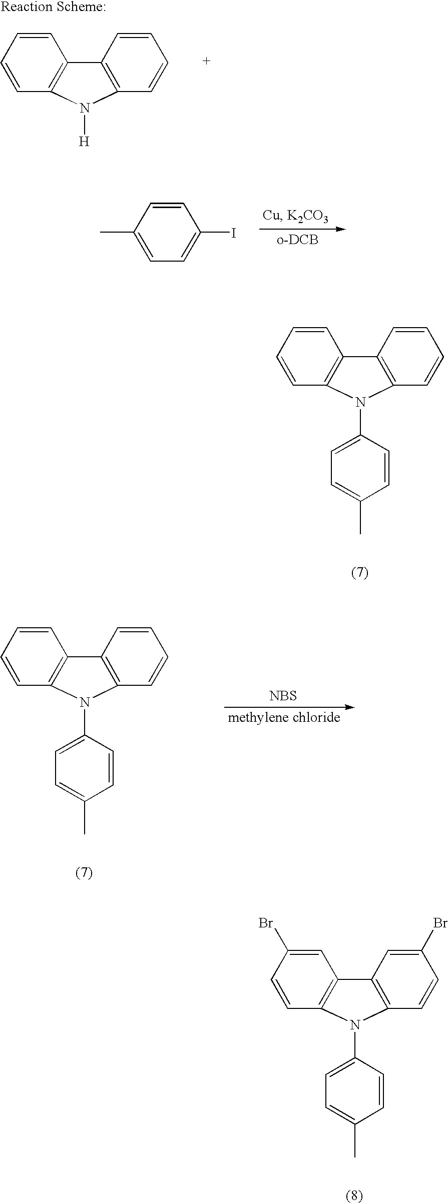

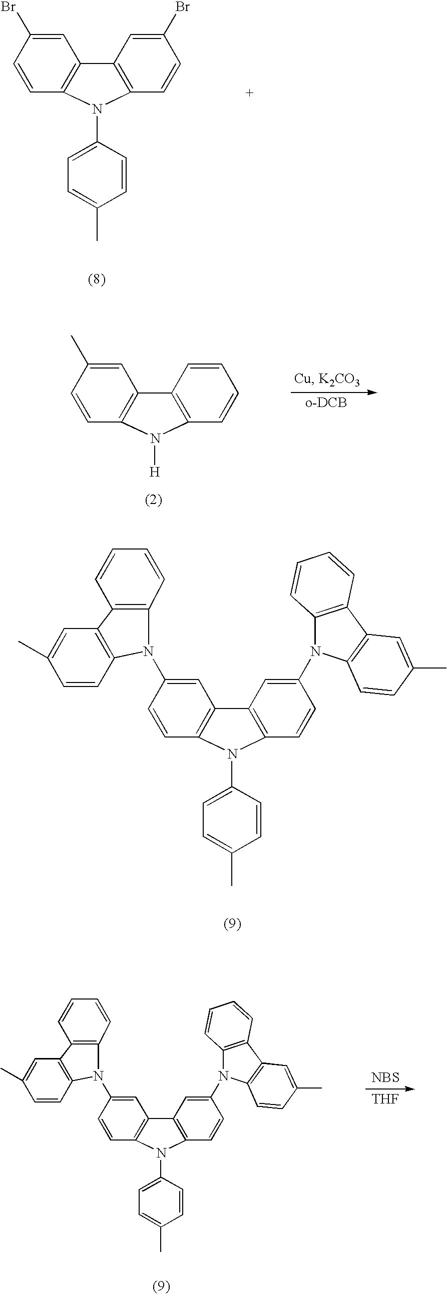

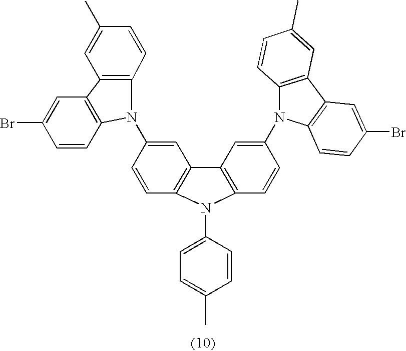

### Synthesis of 9-p-Tolyl-9H-carbazole (Compound 7)

16.7 g (0.1 mol) of carbazole, 32.7 g (0.15 mol) of 4-iodotoluene, 12.8 g (0.2 mol) of copper, 34.5 g (0.25 mol) of potassium carbonate, and 2.8 g (10 mmol) of 18-crown-6 were dispersed in 500 mL of o-dichlorobenzene under nitrogen in a 1 L 3-neck RBF equipped with condenser and Dean Stark trap. The suspension was degassed with nitrogen for 30 minutes and then heated to reflux under nitrogen. In the first several hours of the reaction, there was some water collected in the Dean Stark trap. The water was removed from the Dean Stark trap. After 22 hours, the reaction was cooled to 70° C., the reaction mixture was filtered through a basic alumina bed (˜2 cm thick) and eluted with toluene. The solvents were removed on a rotary evaporator under reduced pressure to give white solids as the crude product. The product was purified by re-crystallization from the mixture of ethanol and toluene. The yield was 15.25 g. The purity was 99.81% by HPLC.

### 3,6-Dibromo-9-p-tolyl-9H-carbazole (Compound 8)

To a solution of 15.02 g (58.3 mmol) of 9-p-tolyl-9H-carbazole (Compound 7) in 170 mL of dichloromethane in a 500 mL 3-neck RBF was added 20.81 g (116.6 mmol) of N-bromosuccinimide in small portions at 0° C. (cooled in ice-water bath). After the addition, the reaction mixture was allowed to warm up to room temperature and was stirred at room temperature overnight. The solvent was then removed on a rotary evaporator and the crude product was purified by re-crystallization from a mixture of ethanol and hexanes. The yield was 18.68 g. The purity was 99.7% by HPLC

### 3,6-Bis(3′-methylcarbazol-9′-yl)-9-p-tolyl-9H-carbazole (Compound 9)

7.25 g (40 mmol) of 3-methylcarbazole (Compound 2), 6.23 g (15 mmol) of 3,6-dibromo-9-p-tolyl-9H-carbazole (8), 6.4 g (0.1 mmol) of copper, 28 g (0.22 mol) of potassium carbonate, and 1.3 g (5.5 mmol) of 18-crown-6 were dispersed in 250 mL of o-dichlorobenzene under nitrogen in a 500 mL 3-neck RBF equipped with condenser and Dean Stark trap and the mixture was heated to reflux. After 4.5 days, an additional 1 g of 3-methylcarbazole was added, and the reflux was continued for another one day. The reaction was then cooled to near room temperature and the reaction mixture was passed through a basic alumina bed (˜1 inch), eluted with ˜300 mL of 1,2-dichlorobenzene. After the removal of most of the solvent, the product was precipitated in methanol. The brown solids were re-dissolved in a small amount of toluene and purified on a silica gel column eluted with hexane+toluene (7:3 in volume). 6.2 g of white solids were obtained and HPLC showed a purity of 98.85%. The white solids were re-crystallized from a mixture of toluene and acetonitrile to give 5.7 g of white solids having a purity of 99.1% as determined by HPLC.

### 3,6-Bis(3′-bromo-6′-methylcarbazol-9′-yl)-9-p-tolyl-9H-carbazole (Compound 10)

To a solution of 5.5 g (8.73 mmol) of 3,6-Bis(3′-methylcarbazol-9′-yl)-9-p-tolyl-9H-carbazole (Compound 9) dissolved in 200 mL of THF was added 3.26 g (18.31 mmol) of N-bromosuccinimide (NBS) dissolved in ˜10 mL of DMF at room temperature. The reaction mixture was stirred at room temperature overnight. The reaction mixture was concentrated to ˜50 mL on a rotary evaporator and the product was precipitated into methanol (300 mL). The crude product was collected as white solids by filtration and then was purified by re-crystallization from a mixture of toluene and ethanol to give the final product as white crystals. The yield was 5.6 g. The purity was 99.8% by HPLC.

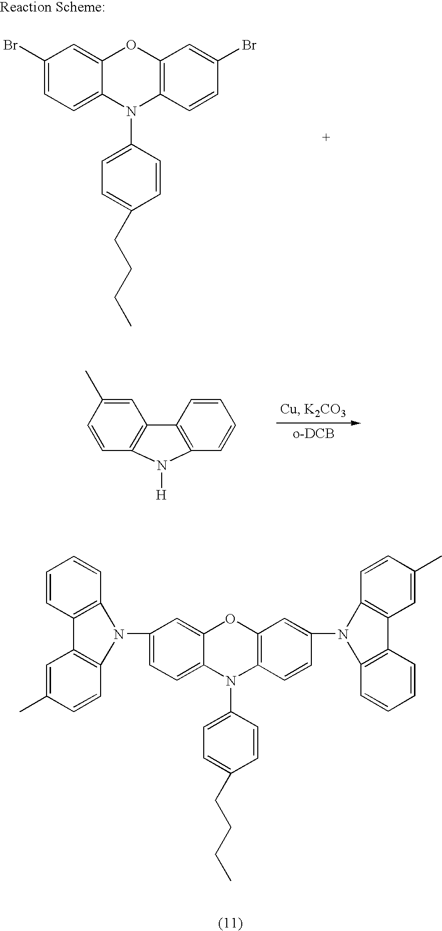

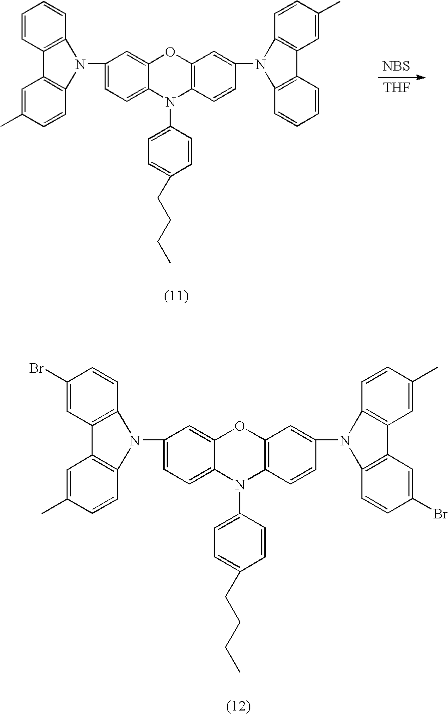

### 3,7-Bis(3′-methylcarbazol-9′-yl)-N-(4-n-butylphenyl)phenoxazine (Compound 11)

7.1 g (15 mmol) of 3,7-dibromo-N-(4-n-butylphenyl)phenoxazine, 8.14 g of 3-methylcarbazole, 7.1 g of copper, 30.4 g of potassium carbonate, 1.45 g of 18-crown-6 were dispersed in 250 ml of o-dichlorobenzene under nitrogen in a 500 ml of 3-neck RFB equipped with condenser and Dean-Stark trap. The suspension was degassed with flowed nitrogen for 15 min. and then was heated to reflux for 7 days. The reaction was allowed to cool to ˜80° C. and was filtered through a basic alumina layer (˜2 cm) and washed with ˜300 ml of o-dichlorobenzene. The combined solutions were evaporated to remove solvent. The crude product was purified by flush chromatography on silica gel eluted with the mixture of toluene and hexanes (2:8 v/v) to give 5.83 grams of the final product as white powders at the purity of 99.3% as indicated by HPLC.

### 3,7-Bis(3′-bromo-6′-methylcarbazol-9′-yl)-N-(4-n-butylphenyl)phenoxazine (Compound 12)

3.35 g (5 mmol) of 3,7-bis(3′-methylcarbazol-9′-yl)-N-(4-n-butylphenyl)phenoxazine dissolved in 200 ml of THF was added 1.8 g (10.1 mmol) of NBS dissolved in ˜10 ml of DMF at 0° C. (ice bath). The reaction mixture was stirred at room temperature overnight. The solvent was removed on rotary evaporator. The crude product was purified by re-crystallization from the mixture of toluene and ethanol to give the final product as white needle crystals. Yield: 2.7 g. Purity: 99.8% (HPLC).

### Preparation of Polymer 1—Blue Light-Emitting Polymer

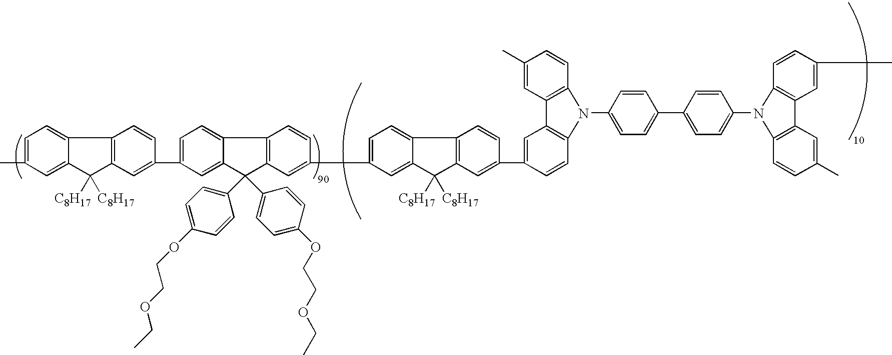

2,7-Bis(1,3,2-dioxaborolan-2-yl)-9,9-dioctylfluorene (2.439 g, 4.575 mmol), 2,7-dibromo-9,9-bis(4-(2-ethoxyethoxy)phenyl)fluorene (2.665 g, 4.077 mmol), 4,4′-bis(3-bromo-6-methylcarbazol-9-yl)biphenyl (0.305 g, 0.453 mmol), Aliquat™ 336 phase transfer agent (0.72 g), and trans-dichloro-bis(triphenylphosphine)palladium (II) (4.2 mg) were dissolved in toluene (40 mL) with stirring in a 250 mL 3-necked flask at room temperature. The reaction mixture was then heated to reflux, whereupon sodium carbonate (2 M, 10 mL) was added. The mixture was stirred for about 2.5 hours, then phenylboronic acid was added (0.27 g), followed by toluene (25 mL), and the reaction mixture was stirred and heated overnight, then allowed to cool. The aqueous phase was separated from the reaction mixture and the organic phase was washed with additional water (100 mL), then added to an aqueous solution of sodium diethyldithiocarbamate trihydrate (DDC, 3 g dissolved in 30 mL water) and heated and stirred under nitrogen at 85° C. for 4 hours. The aqueous phase (16 mL) was separated from the polymer solution, and the organic solution was washed with 2 percent v/v aqueous acetic acid (2×˜100 mL), followed by water washings (3×˜100 mL). The organic phase containing the polymer product was passed through a column of celite (1″), silica (3″), and alumina (1″) and eluted with toluene. The polymer fractions were combined and the solution concentrated in vacuo to produce about a 3 percent w/v solution of polymer in toluene. The product was precipitated into methanol. The polymer was dried overnight in vacuo at 60° C. The polymer was re-dissolved in toluene (170 mL), and then reprecipitated in methanol. The polymer was collected and dried in vacuo as before to yield 3.9 g of white fibers as the final polymer. GPC analysis of the polymer showed a number average molecular weight (Mₙ) of 109,000, a weight average molecular weight (M_{w}) of 210,000, and a polydispersity (M_{w}/Mₙ) of 1.92.

### Preparation of Polymer 2—Blue Light-Emitting Polymer

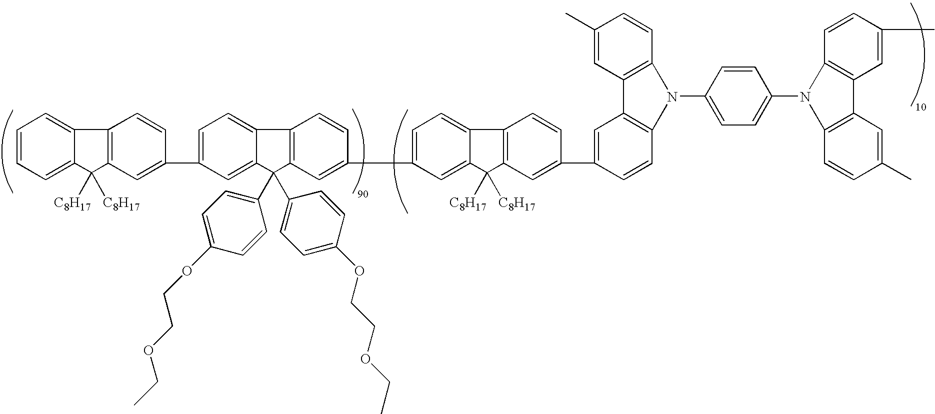

2,7-Bis(1,3,2-dioxaborolan-2-yl)-9,9-dioctylfluorene (2.678 g, 5.05 mmol), 2,7-dibromo-9,9-bis(4-(2-ethoxyethoxy)phenyl)fluorene (2.935 g, 4.50 mmol), 4′,4-bis(3-bromo-6-methylcarbazol-9-yl)benzene (0.297 g, 0.50 mmol), Aliquat™ 336 phase transfer agent (0.6 g), and trans-dichloro-bis(triphenylphosphine)palladium (II) (3 mg) were dissolved in toluene (40 mL) with stirring in a 250 mL 3-necked flask at room temperature. The reaction mixture was then heated to reflux, whereupon sodium carbonate (2 M, 13 mL) was added. The mixture was stirred for about 2.5 hours, then phenylboronic acid was added (0.25 g), followed by toluene (40 mL), and the reaction mixture was stirred and heated overnight, then allowed to cool. An aqueous solution of sodium diethyldithiocarbamate trihydrate (DDC, 3 g dissolved in 30 mL water) was added and the mixture was heated and stirred under nitrogen at 95° C. for 6 hours. The aqueous phase was separated from the polymer solution, and the solution was washed with 2 percent v/v aqueous acetic acid (3×˜300 mL), followed by water washings (300 mL). The polymer solution was poured into stirred methanol (3 L) to precipitate the polymer. The polymer was collected by filtration and dried in vacuum oven at 60 degrees Celsius overnight. The polymer was re-dissolved in 300 mL of toluene and the solution was passed through a column of celite (1″), silica (3″), and alumina (1″) and eluted with toluene. The polymer fractions were combined and the solution concentrated in vacuo to produce about a 3 percent w/v solution of polymer in toluene. The product was precipitated in methanol. The polymer was dried overnight in vacuo at 60 degrees Celsius. The polymer was re-dissolved in toluene (200 mL), and then re-precipitated in methanol. Fibers were collected and dried in vacuo as before to yield 3.5 g of polymer as white fibers. GPC analysis of the polymer showed a number average molecular weight (Mₙ) of 103,867, a weight average molecular weight (M_{w}) of 303,412, and a polydispersity (M_{w}/Mₙ) of 2.92.

### Preparation of Polymer 3—Phosphorescent Light-Emitting Polymer

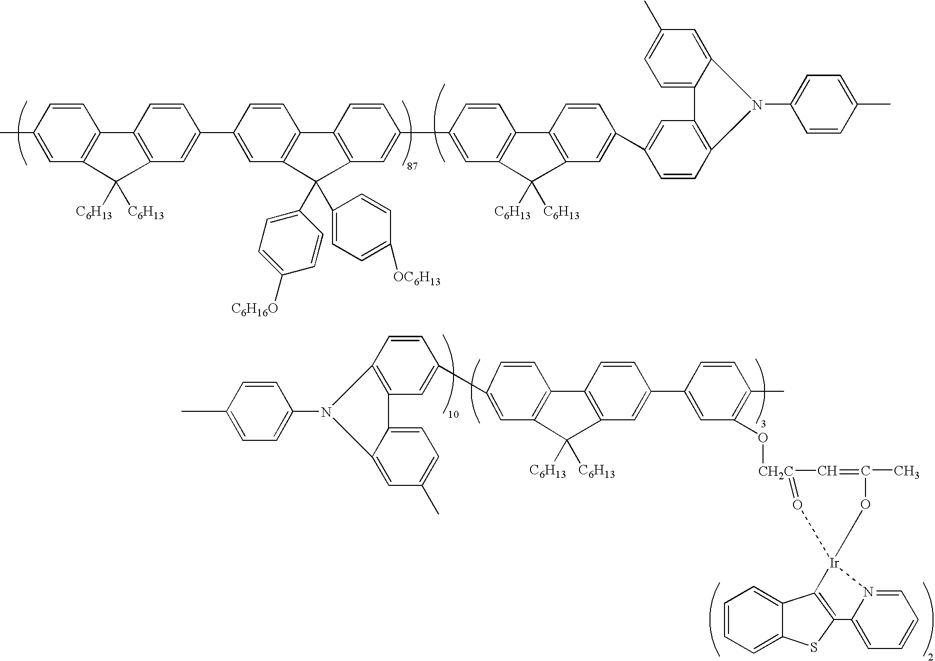

2,7-Bis(1,3,2-dioxaborolan-2-yl)-9,9-dihexylfluorene (1.927 g, 4.08 mmol), 2,7-dibromo-9,9-di(4-hexyloxyphenyl)fluorene (2.359 g, 3.48 mmol), 4,4′-bis(3-bromo-6-methylcarbazol-9-yl)biphenyl (0.271 g, 0.40 mmol), an iridium complex monomer with the structure shown below (0.116 g, 0.12 mmol), Aliquat™ 336 phase transfer agent (0.9 g), and trans-dichloro-bis(triphenylphosphine)palladium (II) (3.6 mg) were dissolved toluene (40 mL) with stirring in a 250 mL 3-necked flask at room temperature. The reaction mixture was then heated to reflux, whereupon sodium carbonate (2 M, 11 mL) was added. The mixture was stirred under nitrogen overnight, then phenylboronic acid was added (0.27 g), followed by toluene (10 mL), and the reaction mixture was stirred at 101 degrees Celsius for 3.5 h, then allowed to cool. An aqueous solution of sodium diethyldithiocarbamate trihydrate (DDC, 3 g dissolved in 30 mL water) was added and the mixture was heated and stirred under nitrogen at 95° C. for 3 hours. The aqueous phase was separated from the polymer solution, and the solution was washed with water (5×˜300 mL). The polymer solution was poured into stirred methanol (3 L) to precipitate the polymer. The polymer was collected by filtration and dried in vacuum oven at 60 degrees Celsius overnight. The polymer was re-dissolved in 300 mL of toluene and the solution was passed through a column of celite (1″), silica (3″), and alumina (1″) and eluted with toluene. The polymer fractions were combined and the solution concentrated in vacuo to produce about a 3 percent w/v solution of polymer in toluene. The product was precipitated in methanol. The polymer was dried overnight in vacuo at 60 degrees Celsius. The polymer was re-dissolved in toluene (200 mL), and then re-precipitated in methanol. The polymer was collected and dried in vacuo as before to yield 2.9 g of the polymer as pale yellow fibers. GPC analysis of the polymer showed a number average molecular weight (Mₙ) of 103,867, a weight average molecular weight (M_{w}) of 303,412, and a polydispersity (M_{w}/Mₙ) of 2.92.

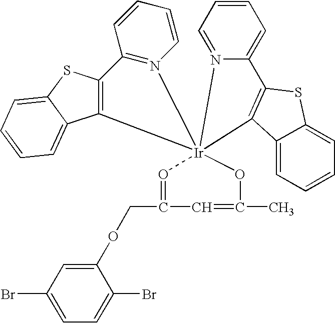

### Preparation of Polymer 4—Blue Light-Emitting Polymer

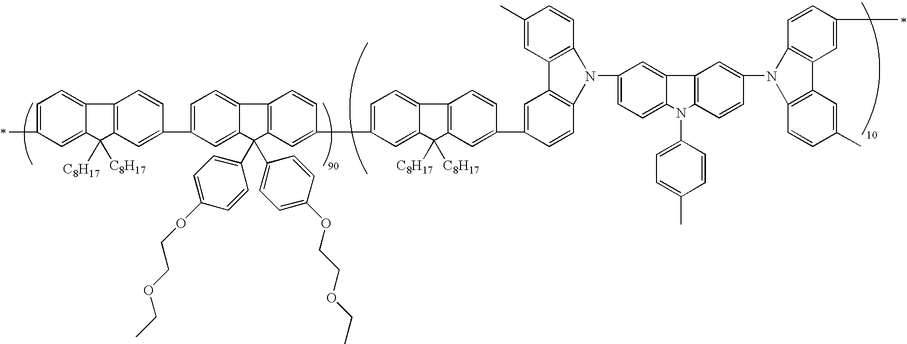

2,7-Bis(1,3,2-dioxaborolan-2-yl)-9,9-dioctylfluorene (2.1426 g, 4.04 mmol), 2,7-dibromo-9,9-bis(4-(2-ethoxyethoxy)phenyl)fluorene (2.3487 g, 3.60 mmol), 3,6-bis(3′-bromo-6′-methylcarbazol-9′-yl)-9-p-tolyl-9H-carbazole (0.3150 g, 0.40 mmol), Aliquat™ 336 phase transfer agent (0.6 g), and trans-dichloro-bis(triphenylphosphine)palladium (II) (2.5 mg) were dissolved in toluene (40 mL) with stirring in a 250 mL 3-necked flask at room temperature. The reaction mixture was then heated to reflux, whereupon sodium carbonate (2 M, 11 mL) was added. The mixture was stirred for about 2.5 hours, then phenylboronic acid was added (0.25 g), followed by toluene (40 mL), and the reaction mixture was stirred and heated overnight, then allowed to cool. An aqueous solution of sodium diethyldithiocarbamate trihydrate (DDC, 3 g dissolved in 30 mL water) was added and the mixture was heated and stirred under nitrogen at 95° C. for 6 hours. The aqueous phase was separated from the polymer solution, and the solution was washed with 2 percent v/v aqueous acetic acid (3×˜300 mL), followed by water washings (300 mL). The polymer solution was poured into stirred methanol (3 L) to precipitate the polymer. The polymer was collected by filtration and dried in vacuum oven at 60 degrees Celsius overnight. The polymer was re-dissolved in 300 mL of toluene and the solution was passed through a column of celite (1″), silica (3″), and alumina (1″) and eluted with toluene. The polymer fractions were combined and the solution concentrated in vacuo to produce about a 3 percent w/v solution of polymer in toluene. The product was precipitated in methanol. The polymer was dried overnight in vacuo at 60 degrees Celsius. The polymer was re-dissolved in toluene (200 mL), and then re-precipitated in methanol. Fibers were collected and dried in vacuo as before to yield 2.6 g of polymer as white fibers. GPC analysis of the polymer showed a number average molecular weight (Mₙ) of 234,239, a weight average molecular weight (M_{w}) of 705,872, and a polydispersity (M_{w}/Mₙ) of 3.01.

### Preparation of Polymer 5A—Blue Light-Emitting Polymer

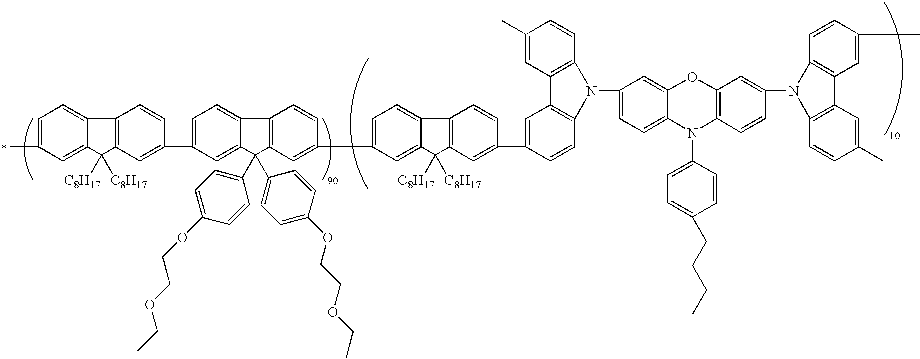

2,7-Bis(1,3,2-dioxaborolan-2-yl)-9,9-dioctylfluorene (1.8562 g, 3.035 mmol), 2,7-dibromo-9,9-bis(4-(2-ethoxyethoxy)phenyl)fluorene (2.0551 g, 3.15 mmol), 3,7-bis(3′-bromo-6′-methylcarbazol-9′-yl)-N-(4-n-butylphenyl)phenoxazine (0.2911 g, 0.35 mmol), Aliquat™ 336 phase transfer agent (0.6 g), and trans-dichloro-bis(triphenylphosphine)palladium (II) (2.5 mg) were dissolved in toluene (40 mL) with stirring in a 250 mL 3-necked flask at room temperature. The reaction mixture was then heated to reflux, whereupon sodium carbonate (2 M, 11 mL) was added. The mixture was stirred for about 2.5 hours, then phenylboronic acid was added (0.25 g), followed by toluene (40 mL), and the reaction mixture was stirred and heated overnight, then allowed to cool. An aqueous solution of sodium diethyldithiocarbamate trihydrate (DDC, 3 g dissolved in 30 mL water) was added and the mixture was heated and stirred under nitrogen at 95° C. for 6 hours. The aqueous phase was separated from the polymer solution, and the solution was washed with 2 percent v/v aqueous acetic acid (3×˜300 mL), followed by water washings (300 mL). The polymer solution was poured into stirred methanol (3 L) to precipitate the polymer. The polymer was collected by filtration and dried in vacuum oven at 60 degrees Celsius overnight. The polymer was re-dissolved in 300 mL of toluene and the solution was passed through a column of celite (1″), silica (3″), and alumina (1″) and eluted with toluene. The polymer fractions were combined and the solution concentrated in vacuo to produce about a 3 percent w/v solution of polymer in toluene. The product was precipitated in methanol. The polymer was dried overnight in vacuo at 60 degrees Celsius. The polymer was re-dissolved in toluene (200 mL), and then re-precipitated in methanol. Fibers were collected and dried in vacuo as before to yield 2.5 g of polymer as white fibers. GPC analysis of the polymer showed a number average molecular weight (Mₙ) of 161,244, a weight average molecular weight (M_{w}) of 409,100, and a polydispersity (M_{w}/Mₙ) of 2.54.

### Preparation of Polymer 5B—Alternating Copolymer with Fluorene

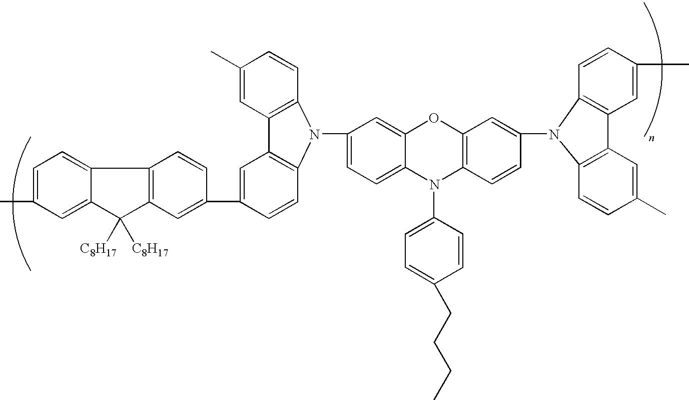

2,7-Bis(1,3,2-dioxaborolan-2-yl)-9,9-dioctylfluorene (1.4850 g, 2.80 mmol), 3,7-bis(3′-bromo-6′-methylcarbazol-9′-yl)-N-(4-n-butylphenyl)phenoxazine (2.3285 g, 2.80 mmol), Aliquat™ 336 phase transfer agent (0.6 g), and trans-dichloro-bis(triphenylphosphine)palladium (II) (2.5 mg) were dissolved in toluene (40 mL) with stirring in a 250 mL 3-necked flask at room temperature. The reaction mixture was then heated to reflux, whereupon sodium carbonate (2 M, 11 mL) was added. The mixture was stirred for about 1 day, then phenylboronic acid was added (0.25 g), followed by toluene (40 mL), and the reaction mixture was stirred and heated overnight, then allowed to cool. An aqueous solution of sodium diethyldithiocarbamate trihydrate (DDC, 3 g dissolved in 30 mL water) was added and the mixture was heated and stirred under nitrogen at 95° C. for 6 hours. The aqueous phase was separated from the polymer solution, and the solution was washed with 2 percent v/v aqueous acetic acid (3×˜300 mL), followed by water washings (300 mL). The polymer solution was poured into stirred methanol (3 L) to precipitate the polymer. The polymer was collected by filtration and dried in vacuum oven at 60 degrees Celsius overnight. The polymer was re-dissolved in 300 mL of toluene and the solution was passed through a column of celite (1″), silica (3″), and alumina (1″) and eluted with toluene. The polymer fractions were combined and the solution concentrated in vacuo to produce about a 3 percent w/v solution of polymer in toluene. The product was precipitated in methanol. The polymer was dried overnight in vacuo at 60 degrees Celsius. The polymer was re-dissolved in toluene (200 mL), and then re-precipitated in methanol. Fibers were collected and dried in vacuo as before to yield 2.2 g of polymer as white fibers. GPC analysis of the polymer showed a number average molecular weight (Mₙ) of 21,072, a weight average molecular weight (M_{w}) of 55,716, and a polydispersity (M_{w}/Mₙ) of 2.64. Following the similar procedure, the following alternating copolymers were made:

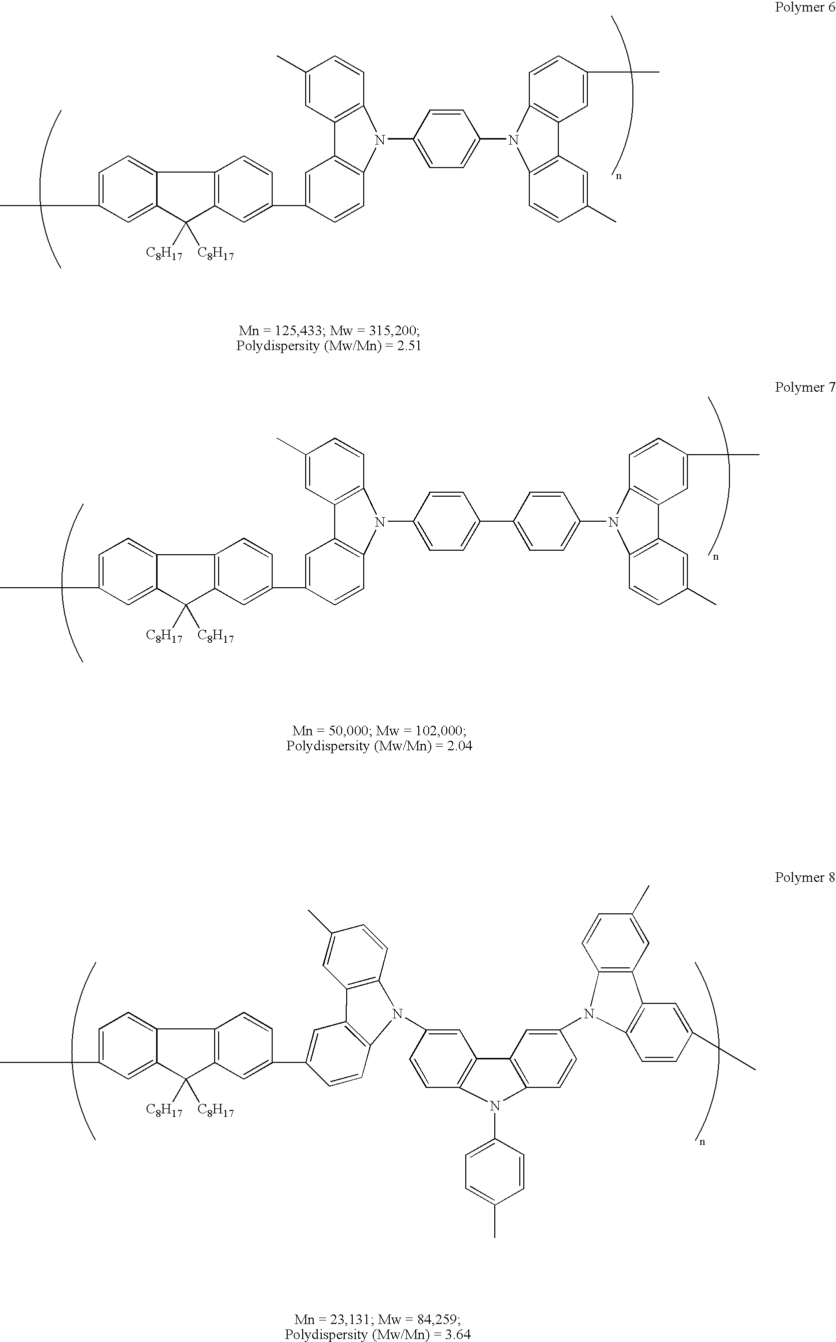

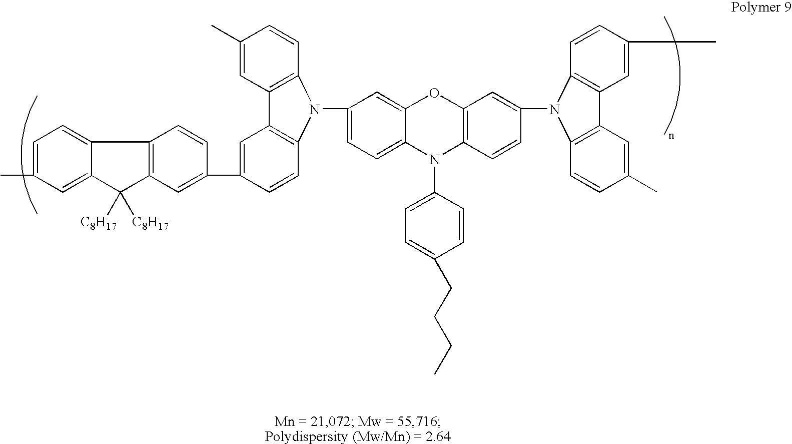

### Blue Polymer LED Devices on Li/Cal/Al Cathode

Polymer 1 (85 mg) was dissolved in xylenes (5 mL) and filtered through a 0.22 μL syringe filter. An 80 nm film of 1:16 w/w polyethylenedioxythiophene (PEDOT):polystyrenesulfonic acid (PSS) was deposited on a cleaned indium-tin-oxide (ITO) coated glass substrate and baked at 200° C. for 15 minutes. An 80 nm film of the polymer/xylenes solution was spin-coated onto the PEDOT:PSS film and the coated substrate was baked at 130° C. under nitrogen for 1 hour. The cathode layers LiF (3 nm), Ca (10 nm), and Al (150 nm) were then vacuum deposited over the polymer film.

Similarly, Polymer 2, Polymer 4, and Polymer 5 were tested using LiF/Ca/Al as the cathode. The typical device data are summarized in the table below.

| Polymers | Efficiency | Voltage | Efficiency | Voltage | Luminance | CIE |
|---|---|---|---|---|---|---|
| (Experiment | (cd/A) @ | (V) @ | (cd/A) @ | (V) @ | (cd/m²) @ | coordinators |
| #) | 400 cd/m² | 400 cd/m² | 1000 cd/m² | 1000 cd/m² | 10 V | (x, y)* |
| Polymer 1 | 1.1 | 4.1 | 1.2 | 5.1 | 3248 | (0.17, 0.12) |
| (02053604) | | | | | | |
| Polymer 2 | 1.0 | 4.2 | 1.1 | 5.3 | 2893 | (0.17, 0.12) |
| (02053604) | | | | | | |
| Polymer 4 | 1.8 | 4.0 | 1.9 | 4.8 | 4819 | (0.17, 0.12) |
| (02053604) | | | | | | |

*Measured at the brightness of 200 cd/m²

As we can see from the data in the table, Polymers 1, 2 and 4 all emitted in deep blue color in the devices using Li/Ca/Al as the cathode.

### Blue Polymer LED Devices on Ba Cathode

Polymer 1 (85 mg) was dissolved in xylenes (5 mL) and filtered through a 0.22 μL syringe filter. An 80 nm film of 1:16 w/w polyethylenedioxythiophene (PEDOT):polystyrenesulfonic acid (PSS) was deposited on a cleaned indium-tin-oxide (ITO) coated glass substrate and baked at 200° C. for 15 minutes. An 80 nm film of the polymer/xylenes solution was spin-coated onto the PEDOT:PSS film and the coated substrate was baked at 130° C. under nitrogen for 1 hour. The cathode metals Ba (5 nm), and Al (150 nm) were then vacuum deposited over the polymer film.

Similarly, Polymer 2, Polymer 4, and Polymer 5 were tested using LiF/Ca/Al as the cathode. The typical device data are summarized in the table below.

| Polymers | Efficiency | Voltage | Efficiency | Voltage | Luminance | CIE |
|---|---|---|---|---|---|---|
| (Experiment | (cd/A) @ | (V) @ | (cd/A) @ | (V) @ | (cd/m²) @ | coordinators |
| #) | 400 cd/m² | 400 cd/m² | 1000 cd/m² | 1000 cd/m² | 10 V | (x, y)* |
| Polymer 1 | 0.3 | 5.5 | 0.4 | 7.9 | 1004 | (0.159 0.081) |
| (02053603) | | | | | | |
| Polymer 2 | 0.4 | 5.5 | 0.4 | 7.7 | 1108 | (0.159, 0.084) |
| (02053603) | | | | | | |
| Polymer 4 | 1.2 | 4.6 | 1.2 | 5.6 | 3378 | (0.164, 0.114) |
| (02053603) | | | | | | |
| Polymer 5A | 1.3 | 7.0 | 1.2 | 8.9 | 1480 | (0.161, 0.129) |
| (03053690) | | | | | | |

*Measured at the brightness of 200 cd/m²

As we can see from the data in the table, Polymers 1, 2, 4, and 5 all emitted in deep blue color in the devices using Ba/Al as the cathode.

### Blue Polymer LED Device Using a Blend Comprising Polymer 6 as Emissive Layer:

17 mg of Polymer 6 and 68 mg of poly(9,9-dioctylfluorene) were dissolved in 5 ml of xylenes and the solution was filtered through a 0.22 μL syringe filter. An 80 nm film of 1:16 w/w polyethylene-dioxythiophene (PEDOT):polystyrenesulfonic acid (PSS) was deposited on a cleaned indium-tin-oxide (ITO) coated glass substrate and baked at 200° C. for 15 minutes. An 80 nm film of the polymer/xylenes solution was spin-coated onto the PEDOT:PSS film and the coated substrate was baked at 130° C. under nitrogen for 1 hour. The cathode layers LiF (3° nm), Ca (10 nm), and Al (150 nm) were then vacuum deposited over the polymer film. The resultant device emitted blue light (CIE coordinates x=0.160; y=0.080) under dc voltage, and produced an average brightness of 400 cd/m² at 7.5 volts with an average light efficiency of 0.2 cd/A. At 10 V, the average brightness of the device was 644 cd/m².

### CONCLUSION

While this invention has been described as having preferred aspects, the instant invention can be further modified within the spirit and scope of this disclosure. This application is, therefore, intended to cover any variations, uses, or adaptations of the present invention using the general principles disclosed herein. Further, this application is intended to cover such departures from the present disclosure as come within the known or customary practice in the art to which this invention pertains and which fall within the limits of the appended claims.

1. A conjugated or partially conjugated polymer comprising a structural unit of Formula I: 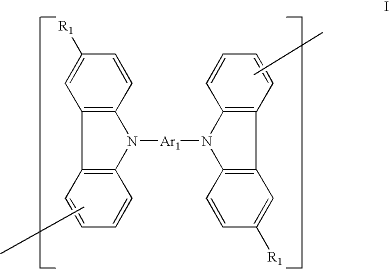
wherein Ar₁ is an aromatic group which contains one or more heteroatoms, or an aromatic group which comprises one or more fused aromatic or non-aromatic rings, said aromatic group may be substituted or unsubstituted; and R₁ is alkyl, alkoxy, an aryl group, cyano, or F.

2. The polymer of claim 1, wherein Ar₁ is selected from the group consisting of fluorenyl, thiophenyl, furanyl, pyrrolyl, pyridinyl, naphthalenyl, anthracenyl, phenanthrenyl, tetracenyl, perylenyl, quinolinyl, isoquinolinyl, quinazolinyl, phenanthridenyl, phenanthrolinyl, phenazinyl, acridinyl, dibenzosilolyl, phthalazinyl, cinnolinyl, quinoxalinyl, benzoxazolyl, benzimidazolyl, benzothiophenyl, benzothiazolyl, carbazolyl, benzoxadiazolyl, benzothiadiazolyl,thieno[3,4-b]pyrazinyl, [1,2,5]thiadiazolo[3,4-g]-quinoxalinyl, benzo[1,2-c; 3-4-c′]bis[1,2,5]-thiadiazolyl, pyrazino[2,3-g]quinoxalinyl, benzofuranyl, indolyl, dibenzofuranyl, dibenzothiophenyl, thianthrenyl, benzodioxinyl, benzodioxanyl, dibenzodioxinyl, phenazinyl, phenoxathiinyl, benzodithiinyl, benzodioxolyl, benzocyclobutenyl, dihydrobenzodithiinyl, dihydrothienodioxinyl, chromanyl, isochromanyl, 9,10 dihydrophenanthrenyl, thiazinyl, phenoxazinyl, isoindolyl, dibenzothiophenesulfonyl, and phenothiazinyl.

3. The polymer of claim 1, wherein Ar₁ is selected from the group consisting of phenyl, biphenyl, a 9,9-disubstituted-2,7-fluorenyl, N-substituted-3,6-carbazolyl, N-substituted-3,7-phenoxazinyl, N-substituted-3,7-phenothiazinyl.

4. A conjugated or partially conjugated polymer comprising a structural unit of Formula I; 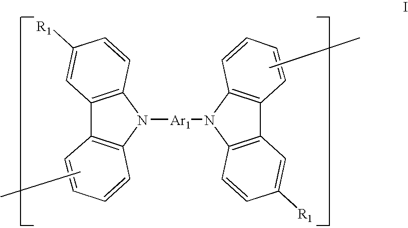
wherein Ar₁ is biphenyl which may be substituted or unsubstituted, and R₁ is alkyl, alkoxy, an aryl group, cyano, or F.

5. The polymer of claim 1 or 4, wherein R₁ is independently alkyl, alkoxyl, an aryl group, cyano, or F.

6. The polymer of claim 1 or 4, wherein R₁ is a C₁-C₂₀ alkyl group, a carbo-C₁-C₂₀-alkoxy group, a C₁-C₂₀-alkoxy group, which may contain one or more heteroatoms, such as O, S, N, or Si, and in which one or more hydrogen atoms may be replaced by F, or aromatic groups, or a C₆-C₄₀ aryl group which may be further substituted and which may contain one or more heteroatoms.

7. The polymer of claim 1 or 4, wherein R₁ is methyl, ethyl, propyl, butyl, methoxy, ethoxy, propoxy, butoxy, phenyl, or tolyl.

8. A conjugated or partially conjugated polymer comprising a structural unit of Formula II: 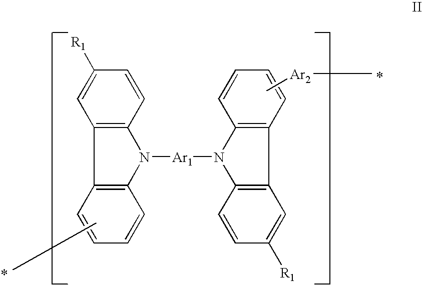
wherein Ar₁ is an aromatic group and Ar₂ is another aromatic group which may be the same or different with Ar₁, both Ar₁ and Ar₂ may be substituted or unsubstituted; Ar₁ contains one or more heteroatomes or comprises one or more fused aromatic or non-aromatic rings, which rings may be substituted or unsubstituted, and R₁ is alkyl, alkoxy, aryl-substituted group, cyano, or F.

9. A conjugated or partially conjugated polymer comprising a structural unit of Formula II; 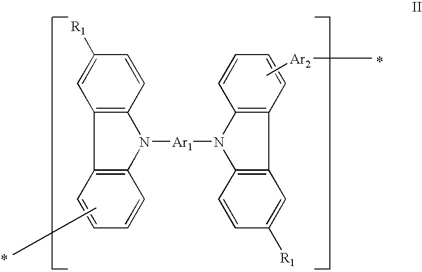
wherein Ar₁ is biphenyl and Ar₂ is another aromatic group which may be the same or different with Ar₁, both Ar₁ and Ar₂ may be substituted or unsubstituted, and R₁ is alkyl, alkoxy, an aryl group, cyano, or F.

10. The polymer of claim 8 or 9, wherein Ar₂ is a 1,4-phenylene, a 1,3-phenylene, a 1,2-phenylene, a 4,4′-biphenylene, a naphthalene-1,4-diyl, a naphthalene-2,6-diyl, a furan-2,5-diyl, a thiophene-2,5-diyl, a 2,2′-bithiophene-5,5-diyl, an anthracene-9,10-diyl, a 2,1,3-benzothiadiazole-4,7-diyl, an N-substituted carbazole-3,6-diyl, an N-substituted carbazole-2,7-diyl, a dibenzosilole-3,8-diyl, a dibenzosilole-4,7-diyl, an N-substituted-phenothiazine-3,7-diyl, an N-substituted-phenoxazine-3,7-diyl, a triarylamine-diyl including a triphenylamine-4,4′-diyl, a diphenyl-p-tolylamine-4,4′-diyl, and an N,N-diphenylaniline-3,5-diyl, an N,N,N′,N′-tetraaryl-1,4-diaminobenzene-diyl, an N,N,N′,N′-tetraarylbenzidine-diyl, an arylsilane-diyl, and an 9,9-disubstituted fluorene-2,7-diyl.

11. The polymer of claim 8 or 9, wherein Ar₂ comprises a fluorene having the Formula III 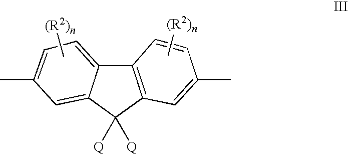
where Q is R′ or Ar, wherein Ar is an aryl or heteroaryl group of C₄ to C₄₀ or substituted aryl or heteroaryl group of C₄ to C₄₀; R′ is independently, in each occurrence, H, C₁₋₄₀ hydrocarbyl or C₃₋₄₀ hydrocarbyl containing one or more S, N, O, P or Si atoms, or both of R′ together with the 9-carbon on the fluorene may form a C₅₋₂₀ ring structure that may contain one or more S, N, Si, P or O atoms; R² is independently in each occurrence a C₁₋₄₀ hydrocarbon, C₃₋₄₀ hydrocarbyl containing one or more heteroatoms of S, N, O, P or Si, or a substituted or unsubstituted aryl group or heteroaryl group; n is independently in each occurrence, 0-3.

12. The polymer of claim 1 or 4 further comprising independently in each occurrence a moiety in the polymer chain selected from the group of conjugated units of the formulas or a combination of the formulas: 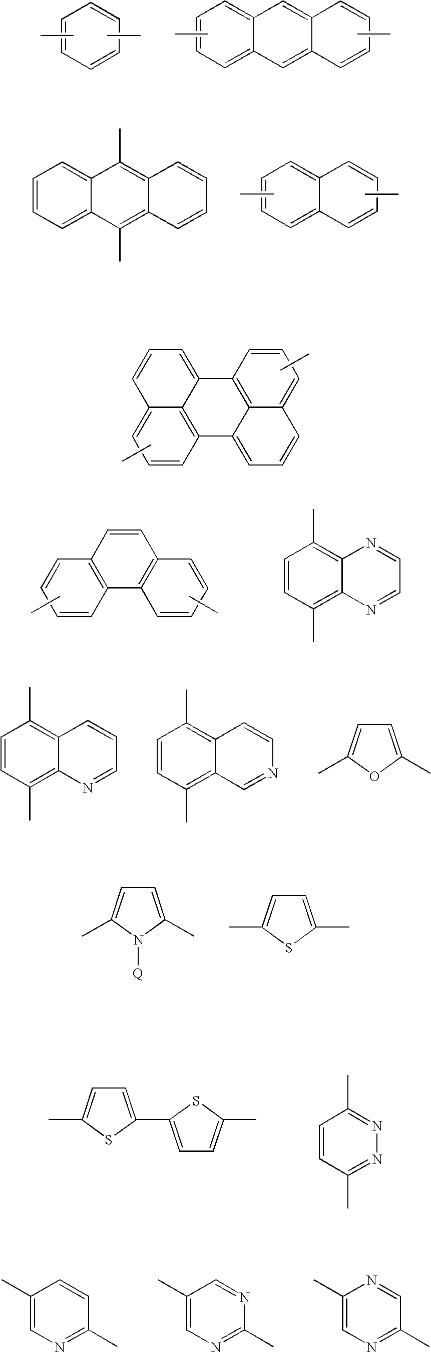 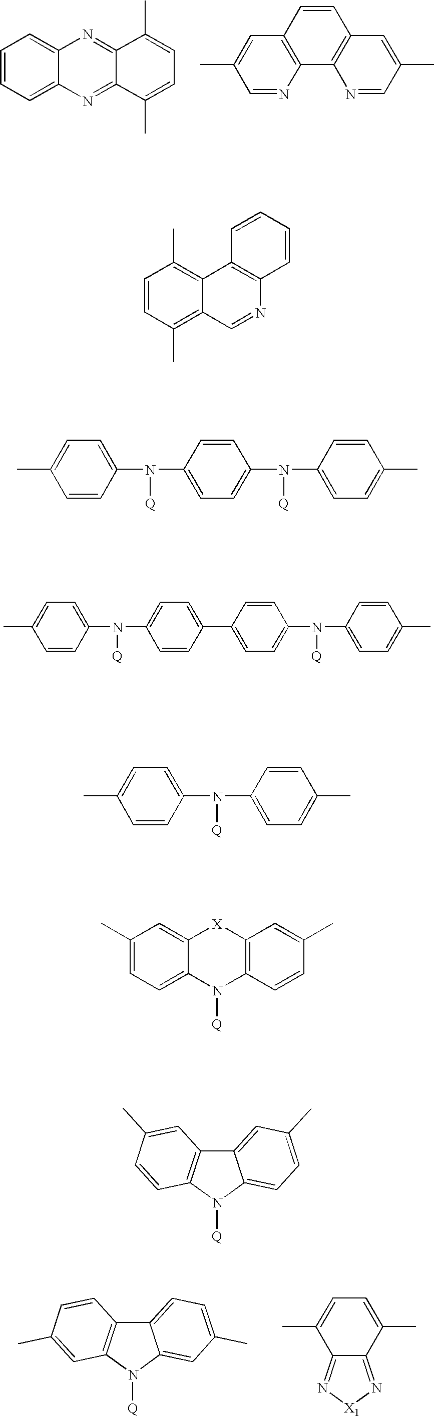 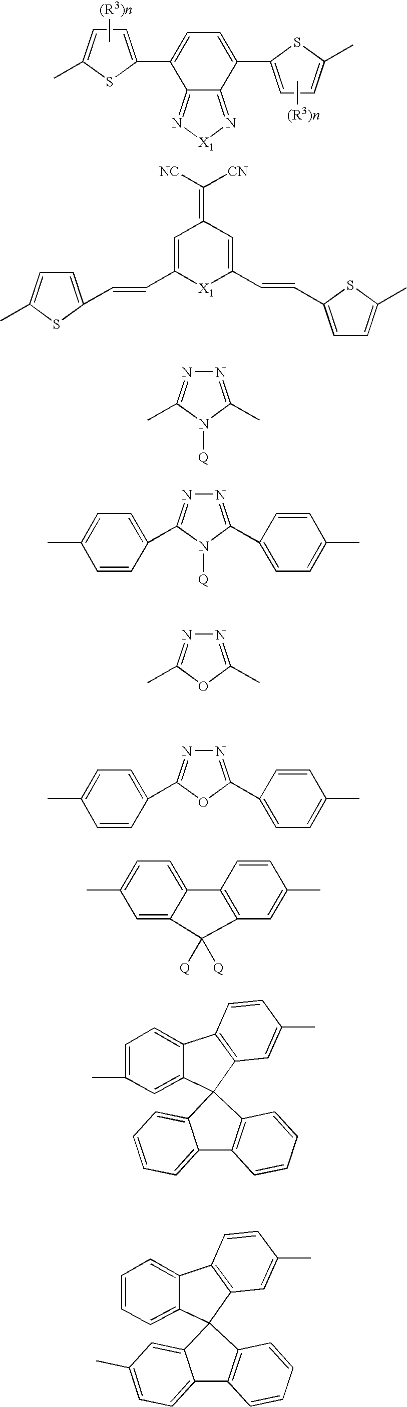 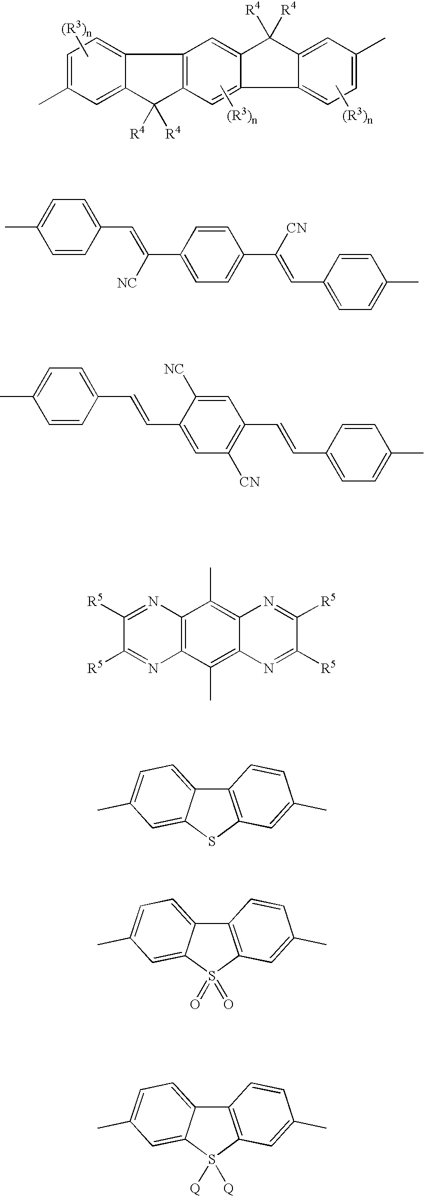
wherein the conjugated units may bear one or more substitutents, such substituents being independently, in each occurrence, C₁₋₂₀ hydrocarbyl, C₁₋₂₀ hydrocarboxyloxy, C₁₋₂₀ thioether, C₁₋₂₀ hydrocarbyloxycarbonyl, C₁₋₂₀ hydrocarboxycarbonyloxy, cyano, or fluoro group;
X₁ is O or S;
Q is R′ or Ar;
R³ is independently, in each occurrence, C₁₋₂₀ hydrocarbyl, C₁₋₂₀ hydrocarbyloxy, C₁₋₂₀ thioether, C₁₋₂₀ hydrocarbyloxycarbonyl, C₁₋₂₀ hydrocarbylcarbonyloxy, cyano or fluoro group; R⁴ is independently, in each occurrence H, C₁₋₄₀ hydrocarbyl or C₃₋₄₀ hydrocarbyl containing one or more S, N, O, P or Si atoms, or both of R⁴ together with the 9-carbon on the fluorene may form a C₅₋₂₀ ring structure that may contain one or more S, N, Si, P or O atoms; and R⁵ is independently, in each occurrence, H, C₁₋₄₀ hydrocarbyl or C₃₋₄₀ hydrocarbyl containing one or more S, N, O, P or Si atoms;
n is independently in each occurrence, 0-3;
Ar is an aryl or heteroaryl group of C₄ to C₄₀ or substituted aryl or heteroaryl group of C₄ to C₄₀; R′ is independently, in each occurrence, H, C₁₋₄₀ hydrocarbyl or C₃₋₄₀ hydrocarbyl containing one or more S, N, O, P or Si atoms, or both of R′ together with the 9-carbon on the fluorene may form a C₅₋₂₀ ring structure that may contain one or more S, N, Si, P or O atoms.

13. The polymer of claim 1, 4, 8 or 9, further comprising a solvent.

14. A film comprising the polymer of claim 1, 4, 8 or 9.

15. The polymer of claim 1, 4, 8 or 9 blended with at least one additional conjugated polymer.

16. The polymer of claim 1, 4, 8 or 9 wherein the polymer emits light in the deep blue range of the spectrum.

17. An electroluminescent device comprising at least one organic film comprising the polymer of claim 1, 4, 8 or 9, arranged between an anode material and a cathode material such that under an applied voltage, the organic film emits blue light which is transmitted through a transparent exterior portion of the device.

18. A field effect transistor comprising:
(a) an insulator layer, the insulator layer being an electrical insulator, the insulator layer having a first side and a second side;
(b) a gate, the gate being an electrical conductor, the gate being positioned adjacent the first side of the insulator layer;
(c) a semiconductor layer, the semiconductor layer comprising the polymer of claim 1, 4, 8 or 9 and a second electrode;
(d) a source, the source being an electrical conductor, the source being in electrical contact with the first end of the semiconductor layer; and
(e) a drain, the drain being an electrical conductor, the drain being in electrical contact with the second end of the semiconductor layer.

19. A photocell comprising a first electrode, a film comprising the polymer of claim 1, 4, 8 or 9 and a second electrode.

20. A compound of Formula IV 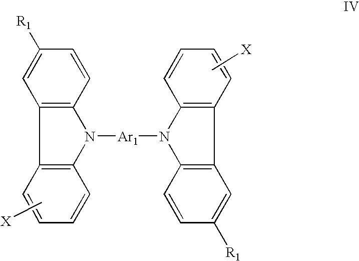
wherein Ar₁ is a substituted or unsubstituted aromatic group which contains one or more heteroatomes or comprises one or more fused aromatic or non-aromatic rings, which rings may be substituted or unsubstituted, R₁ is alkyl, alkoxy, aryl-substituted group, cyano, or F, and X is a halogen or a boronate group.

21. A compound of Formula IV 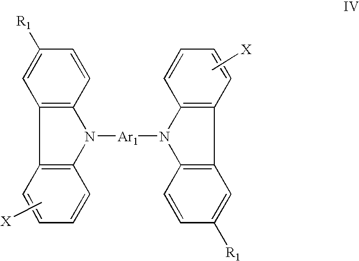
wherein Ar₁ is substituted or unsubstituted biphenyl, R₁, is alkyl, alkoxy, an aryl group, cyano, or F, and X is a halogen or a boronate group.

22. The compound of claim 20 or 21, wherein X is bromine.

23. The compound of claim 20, wherein Ar₁ is selected from the group consisting of fluorenyl, thiophenyl, furanyl, pyrrolyl, pyridinyl, naphthalenyl, anthracenyl, phenanthrenyl, tetracenyl, perylenyl, quinolinyl, isoquinolinyl, quinazolinyl, phenanthridenyl, phenanthrolinyl, phenazinyl, acridinyl, dibenzosilolyl, phthalazinyl, cinnolinyl, quinoxalinyl, benzoxazolyl, benzimidazolyl, benzothiophenyl, benzothiazolyl, carbazolyl, benzoxadiazolyl, benzothiadiazolyl, thieno[3,4-b]pyrazinyl, [1,2,5]thiadiazolo[3,4-g]-quinoxalinyl, benzo[1,2-c; 3-4-c′]bis[1,2,5]-thiadiazolyl, pyrazino[2,3-g]quinoxalinyl, benzofuranyl, indolyl, dibenzofuranyl, dibenzothiophenyl, thianthrenyl, benzodioxinyl, benzodioxanyl, dibenzodioxinyl, phenazinyl, phenoxathiinyl, benzodithiinyl, benzodioxolyl, benzocyclobutenyl, dihydrobenzodithiinyl, dihydrothienodioxinyl, chromanyl, isochromanyl, 9,10-dihydrophenanthrenyl, thiazinyl, phenoxazinyl, isoindolyl, dibenzothiophenesulfonyl, and phenothiazinyl.

24. The compound of claim 20, wherein Ar₁ is selected from the group consisting of phenyl, biphenyl, a 9,9-disubstituted-2,7-fluorenyl, N-substituted-3,6-carbazolyl, N-substituted-3,7-phenoxazinyl, N-substituted-3,7-phenothiazinyl.

25. The compound of claim 20 or 21, wherein R₁ is independently alkyl, alkoxyl, aryl-substituted group, cyano, or F.

26. The compound of claim 20 or 21, wherein R₁ is a C₁-C₂₀ alkyl group, a carbo-C₁-C₂₀-alkoxy group, a C₁-C₂₀-alkoxy group, which may contain one or more heteroatoms, such as O, S, N, P, or Si, and in which one or more hydrogen atoms may be replaced by F, or aromatic groups, or a C₆-C₄₀ aryl group which may be further substituted and which may contain one or more heteroatoms.

27. The compound of claim 20 or 21, wherein R₁ is methyl, ethyl, propyl, butyl, methoxy, ethoxy, propoxy, butoxy, phenyl or tolyl.

28. A conjugated or partially conjugated polymer comprising a structural unit of Formula II: 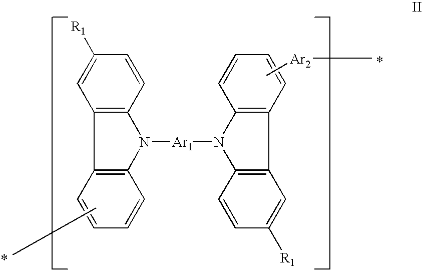
wherein Ar₁ is an aromatic group and Ar₂ is a metal complex monomer, both Ar₁ and Ar₂ may be substituted or unsubstituted, and R₁ is alkyl, alkoxy, aryl group, cyano, or F.

29. A phosphorescent light-emitting polymer comprising the structural unit of Formula I: 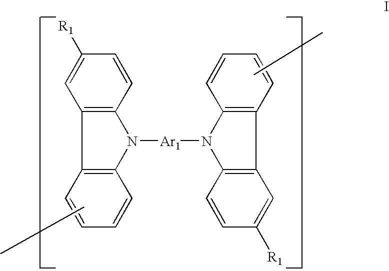
wherein Ar₁ is an aromatic group which may be substituted or unsubstituted, and R₁ is alkyl, alkoxy, an aryl group, cyano, or F.
